SEQUENCE LISTING (2 sequences)

SEQ ID NO: 1 - protein, 369 aa, Myxoma virus
MKYLVLVLCLTSCACRDIGLWTFRYVYNESDNVVFSPYGLTSALSVLRIAAGGNTKREIDVPESVVEDSDAFLALRELFVDASVPLRPEFTAEFSSRFNTSVQRVTFNSENVKDVINSYVKDKTGGDVPRVLDASLDRDTKMLLLSSVRMKTSWRHVFDPSFTTDQPFYSGNVTYKVRMMNKIDTLKTETFTLRNVGYSVTELPYKRRQTAMLLVVPDDLGEIVRALDLSLVRFWIRNMRKDVCQVVMPKFSVESVLDLRDALQRLGVRDAFDPSRADFGQASPSNDLYVTKVLQTSKIEADERGTTASSDTAITLIPRNALTAIVANKPFMFLIYHKPTTTVLFMGTITKGEKVIYDTEGRDDVVSSV

SEQ ID NO: 2 - protein, 6 aa, Artificial Sequence
  Description of Artificial Sequence Synthetic 6xHis tag
HHHHHH

(19) United States
(12) Patent Application Publication
(10) Pub. No.: US 2023/0173080 A1
(43) Pub. Date: Jun. 8, 2023

(54) COMPOSITION OF IMMUNOMODULATING SERPIN, SERP-1

(71) Applicants: Arizona Board of Regents on behalf of Arizona State University, Scottsdale, AZ (US); Serpass Biologics, Inc., Glendale, AZ (US)

(72) Inventors: Alexandra LUCAS, Tempe, AZ (US); Liqiang ZHANG, Chandler, AZ (US); Jordan R. YARON, Scottsdale, AZ (US); Qiuyun GUO, Tempe, AZ (US); John W. WALLEN, III, Las Vegas, NV (US)

(21) Appl. No.: 17/997,239

(22) PCT Filed: Apr. 26, 2021
(86) PCT No.: PCT/US2021/029235

§ 371 Date: Oct. 26, 2022

Related U.S. Application Data
(60) Provisional application No. 63/017,598, filed on Apr. 29, 2020.

(51) Int. Cl. A61K 47/60; C07K 14/81; A61P 7/04; A61P 11/00

(52) U.S. Cl. CPC A61K 47/60; C07K 14/8132; A61P 7/04; A61P 11/00

(57) ABSTRACT

Disclosed herein, in some embodiments, are modified Serp-1 proteins. The modified Serp-1 protein may include a therapeutic enhancing moiety, and be biologically active. In some cases, the therapeutic enhancing moiety is a water soluble polymer such as polyethylene glycol.

### CROSS-REFERENCE

This application claims the benefit of U.S. Provisional Application Ser. No. 63/017,598 filed Apr. 29, 2020 which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Apr. 6, 2021, is named 58709-701_601_SL.txt and is 3,750 bytes in size.

### BACKGROUND

Inflammatory and immune disorders are becoming increasingly abundant, and may affect a wide variety of persons. Improved therapeutics are needed for treating these disorders.

### SUMMARY

Disclosed herein, in some embodiments, are modified Serp-1 proteins. In some embodiments, the modified Serp-1 protein includes at least one therapeutic enhancing moiety, wherein the modified Serp-1 protein is biologically active. Some embodiments include a polypeptide comprising a sequence having at least 80% sequence identity to SEQ ID NO: 1, or a fragment thereof. In some embodiments, the polypeptide is encoded by a nucleic acid sequence. In some embodiments, the therapeutic enhancing moiety is encoded by the nucleic acid. In some embodiments, the polypeptide comprises one, two, three, four or more amino acid substitutions, insertions, or deletions, wherein the substitutions are with natural or non-naturally encoded amino acids. In some embodiments, the therapeutic enhancing moiety comprises a pharmacokinetic enhancing moiety, a stability enhancing moiety, a thermal stability enhancing moiety, or an activity enhancing moiety. In some embodiments, the modified Serp-1 protein has enhanced therapeutic effects, enhanced pharmacokinetics, enhanced stability, enhanced thermal stability, or enhanced activity, compared to an unmodified or wild-type Serp-1 protein. In some embodiments, the therapeutic enhancing moiety comprises a hydrophilic molecule, a PEGylation, an acyl group, a lipid, an alkyl group, a carbohydrate, a polypeptide, a polynucleotide, a polysaccharide, an antibody or antibody fragment, a sialic acid, a prodrug, a serum albumin, an XTEN molecule, an Fc molecule, adnectin, fibronectin, a biologically active molecule, or a water soluble polymer, or a combination thereof. In some embodiments, the therapeutic enhancing moiety is a water soluble polymer comprising polyethylene glycol, polyethylene glycol propionaldehyde, mono C1-C10 alkoxy or an aryloxy derivative thereof, polyethylene glycol, polyvinyl pyrrolidone polyvinyl alcohol, a polyamino acid, divinylether maleic anhydride, N-(2-Hydroxypropyl)-methacrylamide, dextran, a dextran derivative, dextran sulfate, polypropylene glycol, polypropylene oxide copolymer, polyoxyethylated polyol, heparin, a heparin fragment, a polysaccharide, an oligosaccharide, a glycan, cellulose, a cellulose derivative, methylcellulose, carboxymethyl cellulose, starch, a starch derivative, a polypeptide, polyalkylene glycol or a derivative thereof, a copolymer of polyalkylene glycol or a derivative thereof, a polyvinyl ethyl ether, or alpha-beta-poly[(2-hydroxyethyl)-DL-aspartamide, or a combination thereof. In some embodiments, the therapeutic enhancing moiety comprises or consists of a water soluble polymer. In some embodiments, the therapeutic enhancing moiety comprises or consists of polyethylene glycol (PEG). In some embodiments, the PEG is branched. In some embodiments, the PEG is unbranched. In some embodiments, the therapeutic enhancing moiety comprises at least one acyl group, or at least one alkyl group. In some embodiments, the therapeutic enhancing moiety has a molecular weight of about 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, 150 Da, 100 Da, 75 Da, or 57 Da, or a range of molecular weights defined by any two of the aforementioned molecular weights. In some embodiments, the at least one therapeutic enhancing moiety has a molecular weight of about 5 kDa. In some embodiments, at least one therapeutic enhancing moiety has a molecular weight of about 10 kDa. In some embodiments, the therapeutic enhancing moiety is conjugated to a naturally occurring or non-naturally occurring amino acid of the polypeptide. In some embodiments, the therapeutic enhancing moiety is linked to a lysine of the polypeptide. In some embodiments, the therapeutic enhancing moiety is linked to a cysteine of the polypeptide. In some embodiments, the therapeutic enhancing moiety is chemically conjugated to a site at or near an N-terminus or C-terminus of the polypeptide. In some embodiments, the therapeutic enhancing moiety is linked to an end of the polypeptide. In some embodiments, the therapeutic enhancing moiety is linked to an amino terminus of the polypeptide. In some embodiments, the therapeutic enhancing moiety is linked to a carboxyl terminus of the polypeptide. In some embodiments, the therapeutic enhancing moiety is randomly conjugated to the polypeptide. In some embodiments, the therapeutic enhancing moiety is connected to the polypeptide through a linker. In some embodiments, the therapeutic enhancing moiety comprises at least one additional Serp-1 protein or modified Serp-1 protein. In some embodiments, the therapeutic enhancing moiety is linked to multiple Serp-1 proteins. In some embodiments, the therapeutic enhancing moiety is covalently connected to the polypeptide. In some embodiments, the Serp-1 protein is cross-linked with multiple Serp-1 proteins. In some embodiments, at least one therapeutic enhancing moiety comprises 1, 2, 3, 4, 5, 6, 7, 8, 9, 10, 11, 12, 13, 14, 15, 16, 17, 18, 19, 20, 21, 22, 23, 24, 25, or more therapeutic enhancing moieties, or a range of therapeutic enhancing moieties defined by any two of the aforementioned integers. In some embodiments, the polypeptide is produced by a cell. In some embodiments, the polypeptide is secreted from the cell. In some embodiments, the cell is a prokaryotic cell. In some embodiments, the cell is a eukaryotic cell. In some embodiments, the eukaryotic cell is a mammalian cell. In some embodiments, the cell comprises a cell line. In some embodiments, the cell line comprises a CHO cell. In some embodiments, the cell comprises a human cell. In some embodiments, the modified Serp-1 protein is purified or is substantially pure. In some embodiments, the modified Serp-1 protein is purified from the cell or from cell media. In some embodiments, the modified Serp-1 protein exhibits an in vivo half-life that is greater than an unmodified Serp-1 protein. In some embodiments, the unmodified Serp-1 protein comprises the polypeptide. In some embodiments, the unmodified Serp-1 protein exhibits an in vivo half-life of at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 1 week, at least 2 weeks, at least 3 weeks, at least 4 weeks, or longer. In some embodiments, the in vivo half-life is determined in a subject comprising an animal, a vertebrate, a mammal, a rodent, a dog, a rabbit, a horse, cattle, a cat, a sheep, a chicken, a pig, a primate, a non-human primate, or a human. In some embodiments, the half-life is measured in a mammal. In some embodiments, the half-life is measured in a human. In some embodiments, the modified Serp-1 protein is stable at a temperature of 25° C., 30° C., 35° C., 40° C., 45° C., 50° C., 55° C., 60° C., 65° C., 70° C., 75° C., 80° C., 85° C., 90° C., 95° C., 100° C., or more, or a range of temperatures defined by any two of the aforementioned temperatures. In some embodiments, the stability lasts at least 1 minute, at least 2 minutes, at least 3 minutes, at least 4 minutes, at least 5 minutes, at least 10 minutes, at least 15 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 1 week, at least 2 weeks, at least 3 weeks, at least 4 weeks, or longer. In some embodiments, the modified Serp-1 protein exhibits an in vitro thermal stability that is greater than an unmodified Serp-1 protein. In some embodiments, the unmodified Serp-1 protein comprises the polypeptide. In some embodiments, the modified Serp-1 protein is attached to another biologically active moiety. In some embodiments, the modified Serp-1 protein includes at least one, at least two, or three additions, deletions, or substitutions of amino acids of a mature wild-type Serp-1 protein. In some embodiments, the polypeptide comprises a mature wild-type Serp-1 protein. In some embodiments, the biological activity of the modified Serp-1 protein comprises binding to u-plasminogen activator (uPA). In some embodiments, the binding between the modified Serp-1 protein and uPA comprises a binding affinity with an equilibrium dissociation constant (Kd) below 1 mM, below 750 μM, below 500 μM, below 250 μM, below 200 μM, below 150 μM, below 100 μM, below 75 μM, below 50 μM, a Kd below 45 μM, a Kd below 40 μM, a Kd below 35 μM, a Kd below 30 μM, a Kd below 25 μM, a Kd below 20 μM, a Kd below 15 μM, a Kd below 14 μM, a Kd below 13 μM, a Kd below 12 μM, a Kd below 11 μM, a Kd below 10 μM, a Kd below 9 μM, a Kd below 8 μM, a Kd below 7 μM, a Kd below 6 μM, a Kd below 5 μM, a Kd below 4 μM, a Kd below 3 μM, a Kd below 2 μM, or a Kd below 1 μM. In some embodiments, the modified Serp-1 protein is conjugated to at least one of a label, a dye, a polymer, a water-soluble polymer, a photocrosslinker, a radionuclide, a cytotoxic compound, a drug, an affinity label, a photoaffinity label, a reactive compound, a resin, another polypeptide or protein, a polypeptide analog, an antibody, an antibody fragment, a metal chelator, a cofactor, a fatty acid, a carbohydrate, a polynucleotide, a DNA, an RNA, an antisense polynucleotide, a saccharide, a water-soluble dendrimer, a cyclodextrin, an inhibitory ribonucleic acid, a biomaterial, a nanoparticle, a spin label, a fluorophore, a metal-containing moiety, a radioactive moiety, a functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, a quantum dot, a nanotransmitter, a radionucleotide, a radiotransmitter, or a neutron-capture agent, or a combination thereof.

Disclosed herein, in some embodiments, is a culture medium, or an isolated cell, vector, plasmid, prokaryotic cell, eukaryotic cell, virus, AAV, mammalian cell, yeast, bacterium, or cell-free translation system comprising a modified Serp-1 protein described herein. Disclosed herein, in some embodiments, are compositions comprising the culture medium, or isolated cell, vector, plasmid, prokaryotic cell, eukaryotic cell, virus, AAV, mammalian cell, yeast, bacterium, or cell-free translation system, and a pharmaceutically acceptable carrier. Disclosed herein, in some embodiments, are compositions comprising a modified Serp-1 protein described herein, and a pharmaceutically acceptable carrier. In some embodiments, the pharmaceutically acceptable carrier comprises a buffer. Some embodiments include one or more other active compounds comprising a drug, a vaccine, an antibiotic, an antiviral compound, or an anti-parasitic compound. Disclosed herein, in some embodiments, are methods. Some embodiments of the method include administering the composition to a subject. In some embodiments, the subject an animal, a vertebrate, a mammal, a rodent, a dog, a rabbit, a horse, cattle, a cat, a sheep, a chicken, a pig, a primate, or a non-human primate. In some embodiments, the subject is a mammal. In some embodiments, the subject is a human. Disclosed herein, in some embodiments, are modified Serp-1 proteins or compositions containing a modified Serp-1 protein as described herein, for use as a medicament. Disclosed herein, in some embodiments, are uses of a modified Serp-1 protein or composition containing a modified Serp-1 protein as described herein for the manufacture of a medicament for treating or preventing a disease or disorder.

Disclosed herein, in some embodiments, are expression cassettes. In some embodiments, the expression cassette includes a nucleic acid encoding a modified Serp-1 protein described herein. In some embodiments, the nucleic acid comprises DNA. In some embodiments, the expression cassette is configured for expression in a cell. In some embodiments, the cell comprises a mammalian cell. In some embodiments, the cell is a CHO cell. In some embodiments, the cell is a human cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a depiction of a Serp-1 protein.

FIG. 2A includes an overview of Serp-1 effects on intra- and extra-cellular signaling in accordance with some embodiments.

FIG. 2B includes an overview of Serp-1 effects on intra- and extra-cellular signaling in accordance with some embodiments. FIG. 2B includes a magnified depiction of a part of FIG. 2A.

FIG. 2C includes an overview of Serp-1 effects on intra- and extra-cellular signaling in accordance with some embodiments.

FIG. 3A is a diagram showing a secondary structure of some embodiments of a Serp-1 polypeptide. Some relative locations of lysine residues are indicated in the structure.

FIG. 3B shows an exemplary reaction scheme for lysine-specific PEGylation of Serp-1, resulting in modSerp-1^{m5} (5K-PEGylated).

FIG. 3C shows an exemplary reaction scheme for N-terminal PEGylation of Serp-1, resulting in modSerp-1^{s10} (10K-PEGylated).

FIG. 3D is a graphical depiction of an FPLC trace demonstrating separation of wildtype and 5K-PEGylated Serp-1 reaction products.

FIG. 4 is an immunoblot image showing binding of some non-limiting examples of modified Serp-1 proteins and a wild-type Serp-1 protein to urokinase-type-plasminogen activator (uPA). The figure includes an anti-6×His (“6×His” disclosed as SEQ ID NO: 2) western blot demonstrating preservation of serpin function after PEGylation by Serp-1:uPA complex formation in wild-type and modified Serp-1 proteins.

FIG. 5 is an immunoblot image showing thermal stability of a non-limiting example of a modified Serp-1 protein and a wild-type Serp-1 protein.

FIG. 6A is a graphical overview of pristane-induced diffuse alveolar hemorrhage (DAH) studies performed in C57BL6/J mice.

FIG. 6B includes images of gross pathology of pulmonary hemorrhage at 14 days post-induction of DAH in mice. Prevalence of fulminant DAH or protection is graphically indicated.

FIG. 6C includes images and graphical data for a histologic analysis of DAH Score, indicating progressive protection by wildtype and 5K-PEGylated Serp-1.

FIG. 6D includes images and graphical data for a histologic analysis of hemosiderin-laden macrophage deposition by Prussian Blue staining, indicating reduction by both wildtype and 5K-PEGylated Serp-1.

FIG. 7 includes graphical data related to a macrophage response in accordance with some embodiments.

FIG. 8A includes images related to urokinase plasminogen activator surface receptor (uPAR) and inducible nitric oxide synthase (iNOS) in accordance with some embodiments.

FIG. 8B includes graphical data related to inducible nitric oxide synthase (iNOS) in accordance with some embodiments.

FIG. 8C includes graphical data related to inducible nitric oxide synthase (iNOS) in accordance with some embodiments.

FIG. 9A includes graphical data related to tissue measurements of modified Serp-1 protein measurement in accordance with some embodiments.

FIG. 9B includes image data related to tissue measurements of modified Serp-1 protein measurement in accordance with some embodiments.

FIG. 10 includes graphical data related to Prussian blue staining for lung hemorrhage.

FIG. 11 includes graphical data related to iNOS and Ly6G.

FIG. 12 includes in vivo circulating half-life data for a modified Serp-1 protein.

### DETAILED DESCRIPTION

There is an unmet need for better systemic and locally delivered immune modulating compositions. Nearly 60% of Americans have at least one chronic inflammatory condition, 42% have more than one, and 12% of adults have 5 or more. Many existing drugs target inflammation, but not the inflammation source. Additionally, many existing drugs such as steroid drugs have side effects including increased risk of infection, dermatitis, fluid retention, edema, fat deposits in face, chest, upper back, or stomach, mood change, hypertension, a Cushingoid-like state, stomach ulcers, osteoporosis, impaired wound healing, increased appetite, weight gain, worsening of previously acquired medical conditions, depression, hyperglycemia, adrenal suppression or crisis, and/or cataracts. The methods and compositions described herein are designed to address this unmet need.

Viral factors may be used as immune modulating treatments. Myxoma viruses secrete inflammatory cell inhibitors including serpins. Serp-1 is an immune modulating serpin produced by myxoma viruses. Benefits of using a Serp-1 protein as an immune modulating agent may include the capacity for systemic delivery with a focused effect, little or no toxicity, lack of regulation by naturally developed mammalian host systems, resetting of one or more immune response cascades, and/or a powerful immune modulating effects.

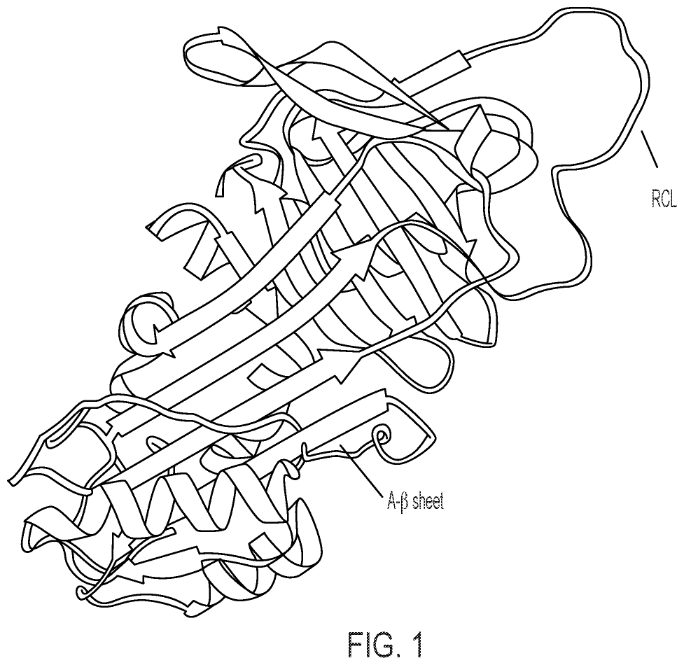

Serp-1, a serine protease inhibitor (serpin), is in some embodiments a secreted, heavily glycosylated 55 kDa protein which was evolved as an immune modulator over millions of years by the rabbit-specific leporipoxvirus, Myxoma virus. FIG. 1 shows a structure of Serp-1 in accordance with some embodiments, with some characteristic serpin features including, but not limited to, an Aβ sheet and a reactive center loop (RCL), which may act as a bait and trap for target proteases. Serp-1 may have immune modulating and pro-resolution activity, and has been explored in animal models, xenograft transplants, balloon angioplasty injury, and atherosclerosis. In a model for rare inflammatory vasculitic syndromes, Serp-1 reduces alveolar hemorrhage and pulmonary consolidation with improved survival, demonstrating the ability for systemically applied Serp-1 to act locally in the lungs. Serp-1 is a “first-in-class” drug and was safe and effective in a Phase IIa trial in patients with acute unstable coronary syndrome after stent implant, with a Major Adverse Cardiac Event (MACE) score of zero and no detected neutralizing antibodies.

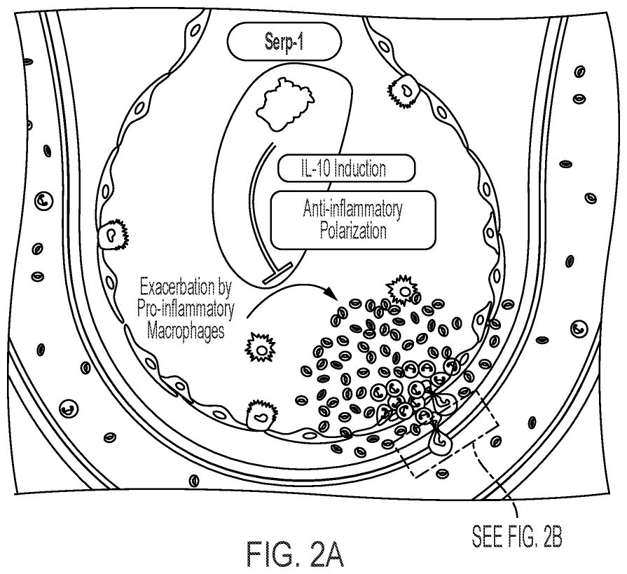

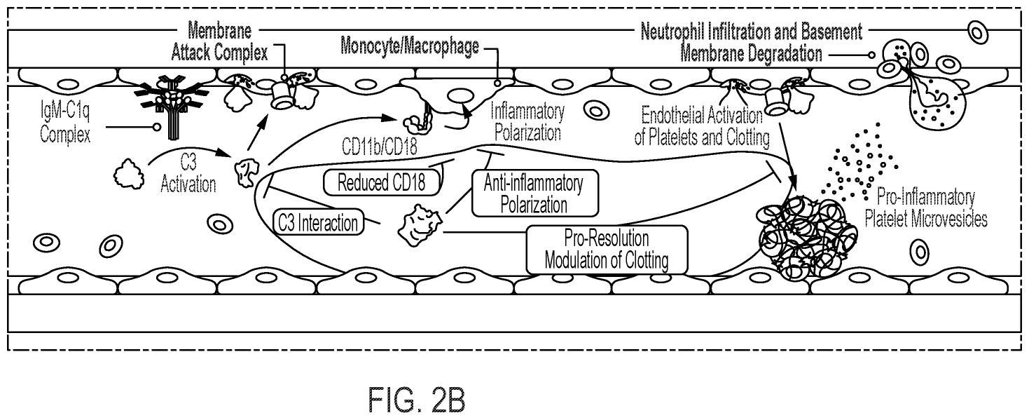

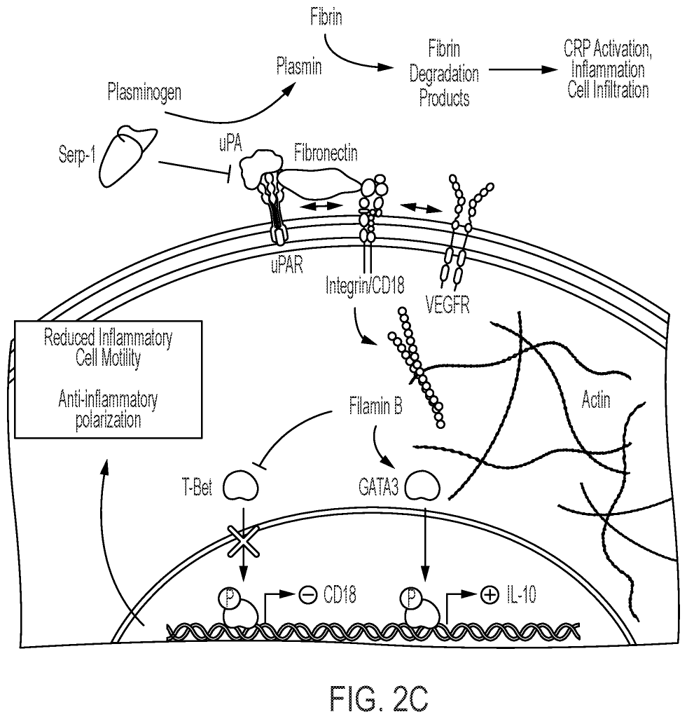

Serpins are a superfamily of proteins that include Serp-1 which may behave as suicide inhibitors by baiting target serine proteases to a recognition sequence in a displayed reactive center loop (RCL). In some cases, when the protease recognized the sequence and initiates digestion, a transient, covalently linked Michaelis complex forms. The formation of the Michaelis complex can destabilize the metastability of the serpin structure, causing the protein to dramatically rearrange by inserting the RCL as the third strand of a 5-strand β-sheet. The target protease is repositioned nearly 70 Å to the opposite pole of the serpin in a denatured, inactive state. Serp-1 canonically targets thrombin, FXa, uPA, tPA and plasmin by a classical serpin mechanism. In addition to the direct effect on its target serine proteases, Serp-1 directly interacts with urokinase-type plasminogen activator receptor (uPAR) and acts by a uPAR-dependent mechanism both in vivo and in vitro. The ability for Serp-1 to reduce plaque growth and transplant vasculopathy in a mouse aortic allograft model was lost in uPAR-deficient grafts and Serp-1-dependent acceleration of full-thickness cutaneous wound healing was lost after treatment with an anti-uPAR neutralizing antibody. Serp-1 may engage the actin-binding protein Filamin B via uPAR and modulates downstream inflammatory signaling resulting in a down-regulation of the C3 receptor component CD18 and inhibition of inflammatory cell migration. In vivo, Serp-1 may promote M2 polarization of macrophages and induce the expression of TL-10 and VEGF as well as some mammalian serpins. As shown in FIG. 2A-2C, Serp-1 may inhibit uPA, limiting fibrin degradation product-induced CRP activation, inflammation and cell infiltration. Serp-1 also may bind to uPAR and the actin binding protein Filamin B, downregulating T-Bet, the transcriptional regulator of CD18/ITGB2, a C3 receptor component. Downregulation of T-Bet may mediate activity of GATA3, resulting in the upregulation of IL-10 and signaling cascades resulting in reduced inflammatory cell motility and in immune modulation and/or pro-resolution polarization. These aspects are examples of biological activities of some Serp-1 proteins.

Modified Serp-1 proteins are useful in a variety of contexts. Co-evolution of viruses with their natural hosts invokes an adaptation arms race, where a successful strategy for the virus relies on immune evasion, often targeting key pathways that drive immune activation. Because viruses are limited in their genomic space, it is common for immune modulating proteins to exhibit multipotent functionality, targeting numerous pathways simultaneously. Translationally, these factors constitute a rich toolbox for developing immune modulators for treating disease.

Serp-1 proteins such as modified Serp-1 proteins have several key advantages. Serp-1 has immune modulating effects. Serp-1 can be delivered systemically with no adverse effects on normal physiology. Serpins have no intrinsic enzymatic activity, thus only acting at the site of active ongoing protease and immune activation and tissue damage. Serp-1 may be highly potent and act at very low doses, thus maintaining a safe treatment window with no influence on naïve immune profiles, and thus maintaining immune competence.

Therapeutic biologics can be engineered to extend half-life and improve bioactivity by the addition of therapeutic enhancing moieties such as those described herein. For example, a Serp-1 protein can be engineered to extend half-life and improve bioactivity by the addition of poly(ethylene glycol) (PEG) moieties through a process called PEGylation. PEGylation is a non-glycoengineering approach in some embodiments that can specifically adjust physicochemical and pharmacokinetic properties of therapeutic proteins such as modified Serp-1 proteins. As disclosed herein, a variety of modified Serp-1 proteins (e.g. PEGylated Serp-1 proteins) have been developed with an aim to improve the pharmacokinetic properties of this already effective immune modulator. Serp-1 can be successfully modified with maintenance of biochemical function, as verified by an in vitro assay, as well as preservation of therapeutic function.

Disclosed herein are compositions comprising a modified Serp-1 protein. The modified Serp-1 protein may include at least one therapeutic enhancing moiety, and be biologically active. In some embodiments, the therapeutic enhancing moiety comprises a water soluble polymer such as polyethylene glycol (PEG). Also provided herein are methods of treatment comprising administering a modified Serp-1 protein to a subject in need thereof.

Disclosed herein are compositions comprising a PEGylated Serp-1 protein. The PEGylated Serp-1 protein may include a polypeptide such as a Serp-1 polypeptide that is covalently linked to at least one polyethylene glycol (PEG). Also provided herein are methods of treatment comprising administering a PEGylated Serp-1 protein to a subject in need thereof.

### I. COMPOSITIONS

Disclosed herein, in some embodiments, are compositions comprising a Serp-1 protein. In some embodiments, the Serp-1 protein is PEGylated. In some embodiments, the Serp-1 protein is modified. In some embodiments, the Serp-1 protein includes at least one therapeutic enhancing moiety. In some embodiments, the Serp-1 protein is biologically active. Some embodiments include a modified Serp-1 protein comprising at least one therapeutic enhancing moiety, wherein the modified Serp-1 protein is biologically active. In some embodiments, a composition described herein is used in a method of treating a disorder in a subject in need thereof. Some embodiments relate to a composition comprising a modified Serp-1 protein for use in a method of treating a disorder as described herein. Some embodiments relate to use of a composition comprising a modified Serp-1 protein, in a method of treating a disorder as described herein. In some embodiments, the Serp-1 protein is secreted. In some embodiments, the Serp-1 protein is glycosylated. In some embodiments, the glycosylation is the same or similar to a wild-type Serp-1 protein.

Disclosed herein, in some embodiments, are modified Serp-1 proteins. In some embodiments, the modified Serp-1 protein includes a polypeptide. In some embodiments, the polypeptide comprises an amino acid sequence. SEQ ID NO: 1 is a non-limiting example of a polypeptide sequence of a Serp-1 protein. Some embodiments include a polypeptide comprising a sequence having at least 80% sequence identity to SEQ ID NO: 1, or a fragment thereof. In some embodiments, the polypeptide is encoded by a nucleic acid. In some embodiments, the therapeutic enhancing moiety is also encoded by the nucleic acid. In some embodiments, the amino acid sequence comprises the sequence of SEQ ID NO: 1. In some embodiments, the amino acid sequence consists of the sequence of SEQ ID NO: 1. In some embodiments, the amino acid sequence is 65%, 70%, 75%, 80%, 85%, 90%, 91%, 92%, 93%, 94%, 95%, 96%, 97%, 98%, 99%, or 100%, or a range of defined by any two of the aforementioned percentages, identical to SEQ ID NO: 1. In some embodiments, the Serp-1 polypeptide is glycosylated in the same or a similar manner as a wild-type Serp-1 protein.

In some embodiments, the amino acid sequence has at least 65% sequence identity to SEQ ID NO: 1, at least 70% sequence identity to SEQ ID NO: 1, at least 75% sequence identity to SEQ ID NO: 1, at least 80% sequence identity to SEQ ID NO: 1, at least 85% sequence identity to SEQ ID NO: 1, at least 90% sequence identity to SEQ ID NO: 1, at least 91% sequence identity to SEQ ID NO: 1, at least 92% sequence identity to SEQ ID NO: 1, at least 93% sequence identity to SEQ ID NO: 1, at least 94% sequence identity to SEQ ID NO: 1, at least 95% sequence identity to SEQ ID NO: 1, at least 96% sequence identity to SEQ ID NO: 1, at least 97% sequence identity to SEQ ID NO: 1, at least 98% sequence identity to SEQ ID NO: 1, or at least 99% sequence identity to SEQ ID NO: 1. In some embodiments, the amino acid sequence has at least 70% sequence identity to a fragment of SEQ ID NO: 1, at least 75% sequence identity to a fragment of SEQ ID NO: 1, at least 80% sequence identity to a fragment of SEQ ID NO: 1, at least 85% sequence identity to a fragment of SEQ ID NO: 1, at least 90% sequence identity to a fragment of SEQ ID NO: 1, at least 91% sequence identity to a fragment of SEQ ID NO: 1, at least 92% sequence identity to a fragment of SEQ ID NO: 1, at least 93% sequence identity to a fragment of SEQ ID NO: 1, at least 94% sequence identity to a fragment of SEQ ID NO: 1, at least 95% sequence identity to a fragment of SEQ ID NO: 1, at least 96% sequence identity to a fragment of SEQ ID NO: 1, at least 97% sequence identity to a fragment of SEQ ID NO: 1, at least 98% sequence identity to a fragment of SEQ ID NO: 1, or at least 99% sequence identity to a fragment of SEQ ID NO: 1.

In some embodiments, the amino acid sequence has no more than 70% sequence identity to SEQ ID NO: 1, no more than 75% sequence identity to SEQ ID NO: 1, no more than 80% sequence identity to SEQ ID NO: 1, no more than 85% sequence identity to SEQ ID NO: 1, no more than 90% sequence identity to SEQ ID NO: 1, no more than 91% sequence identity to SEQ ID NO: 1, no more than 92% sequence identity to SEQ ID NO: 1, no more than 93% sequence identity to SEQ ID NO: 1, no more than 94% sequence identity to SEQ ID NO: 1, no more than 95% sequence identity to SEQ ID NO: 1, no more than 96% sequence identity to SEQ ID NO: 1, no more than 97% sequence identity to SEQ ID NO: 1, no more than 98% sequence identity to SEQ ID NO: 1, no more than 99% sequence identity to SEQ ID NO: 1, or no more than 100% sequence identity to SEQ ID NO: 1. In some embodiments, the amino acid sequence has no more than 70% sequence identity to a fragment of SEQ ID NO: 1, no more than 75% sequence identity to a fragment of SEQ ID NO: 1, no more than 80% sequence identity to a fragment of SEQ ID NO: 1, no more than 85% sequence identity to a fragment of SEQ ID NO: 1, no more than 90% sequence identity to a fragment of SEQ ID NO: 1, no more than 91% sequence identity to a fragment of SEQ ID NO: 1, no more than 92% sequence identity to a fragment of SEQ ID NO: 1, no more than 93% sequence identity to a fragment of SEQ ID NO: 1, no more than 94% sequence identity to a fragment of SEQ ID NO: 1, no more than 95% sequence identity to a fragment of SEQ ID NO: 1, no more than 96% sequence identity to a fragment of SEQ ID NO: 1, no more than 97% sequence identity to a fragment of SEQ ID NO: 1, no more than 98% sequence identity to a fragment of SEQ ID NO: 1, no more than 99% sequence identity to a fragment of SEQ ID NO: 1, or no more than 100% sequence identity to a fragment of SEQ ID NO: 1.

Disclosed herein, in some embodiments, are modified Serp-1 proteins comprising a polypeptide. In some embodiments, the polypeptide comprises one, two, three, four or more amino acid substitutions, insertions, or deletions, wherein the substitutions are with natural or non-naturally encoded amino acids. In some embodiments, the polypeptide comprises at least one amino acid substitution. In some embodiments, the at least one substitution is with natural or non-naturally encoded amino acids. In some embodiments, the substitution is to a different natural amino acid. In some embodiments, the polypeptide comprises at least one amino acid insertion. In some embodiments, the polypeptide comprises at least one amino acid deletion. In some embodiments, the modified Serp-1 protein includes at least one, at least two, or three additions, deletions, or substitutions of amino acids of a mature wild-type Serp-1 protein. In some embodiments, the polypeptide comprises a mature wild-type Serp-1 protein. In some embodiments, the mature wild-type Serp-1 protein comprises a glycoprotein. In some embodiments, the polypeptide comprises a glycoprotein.

Disclosed herein, in some embodiments, are modified Serp-1 proteins comprising a therapeutic enhancing moiety. In some embodiments, the therapeutic enhancing moiety comprises one, two, three, four or more amino acid substitutions, insertions, or deletions, wherein the substitutions are with natural or non-naturally encoded amino acids. In some embodiments, the therapeutic enhancing moiety comprises at least one amino acid substitution. In some embodiments, the at least one substitution is with natural or non-naturally encoded amino acids. In some embodiments, the therapeutic enhancing moiety comprises at least one amino acid insertion. In some embodiments, the therapeutic enhancing moiety comprises at least one amino acid deletion.

Disclosed herein, in some embodiments, are modified Serp-1 proteins comprising a therapeutic enhancing moiety. In some embodiments, the therapeutic enhancing moiety comprises a pharmacokinetic enhancing moiety, a stability enhancing moiety, a thermal stability enhancing moiety, or an activity enhancing moiety. In some embodiments, the therapeutic enhancing moiety includes a pharmacokinetic enhancing moiety. In some embodiments, the therapeutic enhancing moiety includes a stability enhancing moiety. In some embodiments, the therapeutic enhancing moiety includes a thermal stability enhancing moiety. In some embodiments, the therapeutic enhancing moiety includes an activity enhancing moiety.

Disclosed herein, in some embodiments, are modified Serp-1 proteins. In some embodiments, the modified Serp-1 protein has enhanced therapeutic effects, enhanced pharmacokinetics, enhanced stability, enhanced thermal stability, or enhanced activity, compared to an unmodified or wild-type Serp-1 protein. In some embodiments, the modified Serp-1 protein has enhanced therapeutic effects, compared to an unmodified or wild-type Serp-1 protein. In some embodiments, the modified Serp-1 protein has enhanced pharmacokinetics, compared to an unmodified or wild-type Serp-1 protein. In some embodiments, the modified Serp-1 protein has enhanced stability, compared to an unmodified or wild-type Serp-1 protein. In some embodiments, the modified Serp-1 protein has enhanced thermal stability, compared to an unmodified or wild-type Serp-1 protein. In some embodiments, the modified Serp-1 protein has enhanced activity, compared to an unmodified or wild-type Serp-1 protein.

In some embodiments, the at least one therapeutic enhancing moiety is not included as part of a wild-type Serp-1 protein. In some embodiments, the at least one therapeutic enhancing moiety is not included as part of a naturally produced Serp-1 protein.

In some embodiments, the therapeutic enhancing moiety comprises or consists of a polymer. In some embodiments, the therapeutic enhancing moiety comprises a polymer. In some embodiments, the therapeutic enhancing moiety consists of a polymer.

Disclosed herein, in some embodiments, are modified Serp-1 proteins comprising a therapeutic enhancing moiety. In some embodiments, the therapeutic enhancing moiety comprises or consists of a water soluble polymer. In some embodiments, the therapeutic enhancing moiety comprises a water soluble polymer. In some embodiments, the therapeutic enhancing moiety consists of a water soluble polymer. In some embodiments, the therapeutic enhancing moiety is a water soluble polymer comprising polyethylene glycol (PEG), polyethylene glycol propionaldehyde, mono C1-C10 alkoxy or an aryloxy derivative thereof, monomethoxy-polyethylene glycol, polyvinyl pyrrolidone, polyvinyl alcohol, a polyamino acid, divinylether maleic anhydride, N-(2-Hydroxypropyl)-methacrylamide, dextran, a dextran derivative, dextran sulfate, polypropylene glycol, polypropylene oxide copolymer, polyoxyethylated polyol, heparin, a heparin fragment, a polysaccharide, an oligosaccharide, a glycan, cellulose, a cellulose derivative, methylcellulose, carboxymethyl cellulose, starch, a starch derivative, a polypeptide, polyalkylene glycol or a derivative thereof, a copolymer of polyalkylene glycol or a derivative thereof, a polyvinyl ethyl ether, or alpha-beta-poly[(2-hydroxyethyl)-DL-aspartamide, or a combination thereof. In some embodiments, water soluble polymer comprises PEG. In some embodiments, water soluble polymer comprises PEG propionaldehyde. In some embodiments, water soluble polymer comprises mono C1-C10 alkoxy or an aryloxy derivative thereof. In some embodiments, water soluble polymer comprises monomethoxy-polyethylene glycol. In some embodiments, water soluble polymer comprises polyvinyl pyrrolidone. In some embodiments, water soluble polymer comprises polyvinyl alcohol. In some embodiments, water soluble polymer comprises a polyamino acid. In some embodiments, water soluble polymer comprises divinylether maleic anhydride. In some embodiments, water soluble polymer comprises N-(2-Hydroxypropyl)-methacrylamide. In some embodiments, water soluble polymer comprises dextran. In some embodiments, water soluble polymer comprises a dextran derivative. In some embodiments, water soluble polymer comprises dextran sulfate. In some embodiments, water soluble polymer comprises polypropylene glycol. In some embodiments, water soluble polymer comprises a polypropylene oxide copolymer. In some embodiments, water soluble polymer comprises polyoxyethylated polyol. In some embodiments, water soluble polymer comprises heparin. In some embodiments, water soluble polymer comprises a heparin fragment. In some embodiments, water soluble polymer comprises a polysaccharide. In some embodiments, water soluble polymer comprises an oligosaccharide. In some embodiments, water soluble polymer comprises a glycan. In some embodiments, water soluble polymer comprises cellulose. In some embodiments, water soluble polymer comprises a cellulose derivative. In some embodiments, water soluble polymer comprises methylcellulose. In some embodiments, water soluble polymer comprises carboxymethyl cellulose. In some embodiments, water soluble polymer comprises starch. In some embodiments, water soluble polymer comprises a starch derivative. In some embodiments, water soluble polymer comprises a polypeptide. In some embodiments, water soluble polymer comprises polyalkylene glycol or a derivative thereof. In some embodiments, water soluble polymer comprises a copolymer of polyalkylene glycol or a derivative thereof. In some embodiments, water soluble polymer comprises a polyvinyl ethyl ether. In some embodiments, water soluble polymer comprises alpha-beta-poly[(2-hydroxyethyl)-DL-aspartamide. In some embodiments, the water soluble polymer is branched. In some embodiments, the water soluble polymer is unbranched. In some embodiments, the PEG is branched. In some embodiments, the PEG is unbranched. In some embodiments, the water soluble polymer comprises a combination of any of the aforementioned molecules

Disclosed herein, in some embodiments, are modified Serp-1 proteins comprising a therapeutic enhancing moiety. In some embodiments, the therapeutic enhancing moiety comprises a hydrophilic molecule, an acyl group, a lipid, an alkyl group, a carbohydrate, a polypeptide, a polynucleotide, a polysaccharide, an antibody or antibody fragment, a sialic acid, a prodrug, a serum albumin, an XTEN molecule, an Fc molecule, adnectin, fibronectin, a biologically active molecule, or a water soluble polymer, or a combination thereof. In some embodiments, the therapeutic enhancing moiety comprises a hydrophilic molecule. In some embodiments, the therapeutic enhancing moiety comprises an acyl group. In some embodiments, the therapeutic enhancing moiety comprises a lipid. In some embodiments, the therapeutic enhancing moiety comprises an alkyl group. In some embodiments, the therapeutic enhancing moiety comprises a carbohydrate. In some embodiments, the therapeutic enhancing moiety comprises a polypeptide. In some embodiments, the therapeutic enhancing moiety comprises a polynucleotide. In some embodiments, the therapeutic enhancing moiety comprises a polysaccharide. In some embodiments, the therapeutic enhancing moiety comprises an antibody. In some embodiments, the therapeutic enhancing moiety comprises an antibody fragment. In some embodiments, the therapeutic enhancing moiety comprises a sialic acid. In some embodiments, the therapeutic enhancing moiety comprises a prodrug. In some embodiments, the therapeutic enhancing moiety comprises serum albumin. In some embodiments, the therapeutic enhancing moiety comprises an XTEN molecule. In some embodiments, the therapeutic enhancing moiety comprises an Fc molecule. In some embodiments, the therapeutic enhancing moiety comprises adnectin. In some embodiments, the therapeutic enhancing moiety comprises fibronectin. In some embodiments, the therapeutic enhancing moiety comprises a biologically active molecule. In some embodiments, the therapeutic enhancing moiety comprises or consists of a water soluble polymer. In some embodiments, the therapeutic enhancing moiety comprises a combination of any of the aforementioned molecules.

In some embodiments, the therapeutic enhancing moiety comprises at least one acyl group, or at least one alkyl group. In some embodiments, the therapeutic enhancing moiety comprises at least one acyl group. In some embodiments, the therapeutic enhancing moiety comprises at least one alkyl group.

Disclosed herein, in some embodiments, are modified Serp-1 proteins comprising a therapeutic enhancing moiety. In some embodiments, the therapeutic enhancing moiety has a molecular weight of about 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, 150 Da, 100 Da, 75 Da, or 57 Da, or a range of molecular weights defined by any two of the aforementioned molecular weights.

In some embodiments, the therapeutic enhancing moiety has a molecular weight of at least 100,000 Da, a molecular weight of at least 95,000 Da, a molecular weight of at least 90,000 Da, a molecular weight of at least 85,000 Da, a molecular weight of at least 80,000 Da, a molecular weight of at least 75,000 Da, a molecular weight of at least 70,000 Da, a molecular weight of at least 65,000 Da, a molecular weight of at least 60,000 Da, a molecular weight of at least 55,000 Da, a molecular weight of at least 50,000 Da, a molecular weight of at least 45,000 Da, a molecular weight of at least 40,000 Da, a molecular weight of at least 35,000 Da, a molecular weight of at least 30,000 Da, a molecular weight of at least 25,000 Da, a molecular weight of at least 20,000 Da, a molecular weight of at least 15,000 Da, a molecular weight of at least 10,000 Da, a molecular weight of at least 9,000 Da, a molecular weight of at least 8,000 Da, a molecular weight of at least 7,000 Da, a molecular weight of at least 6,000 Da, a molecular weight of at least 5,000 Da, a molecular weight of at least 4,000 Da, a molecular weight of at least 3,000 Da, a molecular weight of at least 2,000 Da, a molecular weight of at least 1,000 Da, a molecular weight of at least 900 Da, a molecular weight of at least 800 Da, a molecular weight of at least 700 Da, a molecular weight of at least 600 Da, a molecular weight of at least 500 Da, a molecular weight of at least 400 Da, a molecular weight of at least 300 Da, a molecular weight of at least 200 Da, a molecular weight of at least 150 Da, a molecular weight of at least 100 Da, a molecular weight of at least 75 Da, or a molecular weight of at least 57 Da.

In some embodiments, the therapeutic enhancing moiety has a molecular weight of no more than 100,000 Da, a molecular weight of no more than 95,000 Da, a molecular weight of no more than 90,000 Da, a molecular weight of no more than 85,000 Da, a molecular weight of no more than 80,000 Da, a molecular weight of no more than 75,000 Da, a molecular weight of no more than 70,000 Da, a molecular weight of no more than 65,000 Da, a molecular weight of no more than 60,000 Da, a molecular weight of no more than 55,000 Da, a molecular weight of no more than 50,000 Da, a molecular weight of no more than 45,000 Da, a molecular weight of no more than 40,000 Da, a molecular weight of no more than 35,000 Da, a molecular weight of no more than 30,000 Da, a molecular weight of no more than 25,000 Da, a molecular weight of no more than 20,000 Da, a molecular weight of no more than 15,000 Da, a molecular weight of no more than 10,000 Da, a molecular weight of no more than 9,000 Da, a molecular weight of no more than 8,000 Da, a molecular weight of no more than 7,000 Da, a molecular weight of no more than 6,000 Da, a molecular weight of no more than 5,000 Da, a molecular weight of no more than 4,000 Da, a molecular weight of no more than 3,000 Da, a molecular weight of no more than 2,000 Da, a molecular weight of no more than 1,000 Da, a molecular weight of no more than 900 Da, a molecular weight of no more than 800 Da, a molecular weight of no more than 700 Da, a molecular weight of no more than 600 Da, a molecular weight of no more than 500 Da, a molecular weight of no more than 400 Da, a molecular weight of no more than 300 Da, a molecular weight of no more than 200 Da, a molecular weight of no more than 150 Da, a molecular weight of no more than 100 Da, a molecular weight of no more than 75 Da, or a molecular weight of no more than 57 Da.

In some embodiments, the therapeutic enhancing moiety has a molecular weight of 5 kDa, 10 kDa, 15 kDa, 20 kDa, 25 kDa, 30 kDa, 35 kDa, or 40 kDa, or a range of molecular weights defined by any two of the aforementioned molecular weights. In some embodiments, the therapeutic enhancing moiety has a molecular weight of about 5 kDa, about 10 kDa, about 15 kDa, about 20 kDa, about 25 kDa, about 30 kDa, about 35 kDa, or about 40 kDa, or a range of molecular weights defined by any two of the aforementioned molecular weights. In some embodiments, the therapeutic enhancing moiety has a molecular weight of 5-40 kDa. In some embodiments, the therapeutic enhancing moiety has a molecular weight of about 5-40 kDa. In some embodiments, the therapeutic enhancing moiety has a molecular weight of 5 kDa. In some embodiments, the therapeutic enhancing moiety has a molecular weight of about 5 kDa. In some embodiments, the therapeutic enhancing moiety has a molecular weight of 10 kDa. In some embodiments, the therapeutic enhancing moiety has a molecular weight of about 10 kDa. In some embodiments, the therapeutic enhancing moiety has a molecular weight of 20 kDa. In some embodiments, the therapeutic enhancing moiety has a molecular weight of about 20 kDa. In some embodiments, the therapeutic enhancing moiety has a molecular weight of 30 kDa. In some embodiments, the therapeutic enhancing moiety has a molecular weight of about 30 kDa. In some embodiments, the therapeutic enhancing moiety has a molecular weight of 40 kDa. In some embodiments, the therapeutic enhancing moiety has a molecular weight of about 40 kDa. In some embodiments, the therapeutic enhancing moiety has a molecular weight of 50 kDa. In some embodiments, the therapeutic enhancing moiety has a molecular weight of about 50 kDa.

Disclosed herein, in some embodiments, is a therapeutic enhancing moiety comprising a water soluble polymer. In some embodiments, the water soluble polymer has a molecular weight of 5 kDa, 10 kDa, 15 kDa, 20 kDa, 25 kDa, 30 kDa, 35 kDa, or 40 kDa, or a range of molecular weights defined by any two of the aforementioned molecular weights. In some embodiments, the water soluble polymer has a molecular weight of about 5 kDa, about 10 kDa, about 15 kDa, about 20 kDa, about 25 kDa, about 30 kDa, about 35 kDa, or about 40 kDa, or a range of molecular weights defined by any two of the aforementioned molecular weights. In some embodiments, the water soluble polymer has a molecular weight of 5-40 kDa. In some embodiments, the water soluble polymer has a molecular weight of about 5-40 kDa. In some embodiments, the water soluble polymer has a molecular weight of 5 kDa. In some embodiments, the water soluble polymer has a molecular weight of about 5 kDa. In some embodiments, the water soluble polymer has a molecular weight of 10 kDa. In some embodiments, the water soluble polymer has a molecular weight of about 10 kDa. In some embodiments, the water soluble polymer has a molecular weight of 20 kDa. In some embodiments, the water soluble polymer has a molecular weight of about 20 kDa. In some embodiments, the water soluble polymer has a molecular weight of 30 kDa. In some embodiments, the water soluble polymer has a molecular weight of about 30 kDa. In some embodiments, the water soluble polymer has a molecular weight of 40 kDa. In some embodiments, the water soluble polymer has a molecular weight of about 40 kDa. In some embodiments, the water soluble polymer has a molecular weight of 50 kDa. In some embodiments, the water soluble polymer has a molecular weight of about 50 kDa.

Disclosed herein, in some embodiments, are modified Serp-1 proteins comprising a polypeptide and a therapeutic enhancing moiety. In some embodiments, the modified Serp-1 protein is attached to another biologically active moiety.

Disclosed herein, in some embodiments, are modified Serp-1 proteins comprising a polypeptide and a therapeutic enhancing moiety. In some embodiments, the modified Serp-1 protein is conjugated to at least one of a label, a dye, a polymer, a water-soluble polymer, a photocrosslinker, a radionuclide, a cytotoxic compound, a drug, an affinity label, a photoaffinity label, a reactive compound, a resin, another polypeptide or protein, a polypeptide analog, an antibody, an antibody fragment, a metal chelator, a cofactor, a fatty acid, a carbohydrate, a polynucleotide, a DNA, an RNA, an antisense polynucleotide, a saccharide, a water-soluble dendrimer, a cyclodextrin, an inhibitory ribonucleic acid, a biomaterial, a nanoparticle, a spin label, a fluorophore, a metal-containing moiety, a radioactive moiety, a functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, a quantum dot, a nanotransmitter, a radionucleotide, a radiotransmitter, or a neutron-capture agent, or a combination thereof.

Disclosed herein, in some embodiments, are modified Serp-1 proteins comprising a polypeptide and a therapeutic enhancing moiety. In certain embodiments, the polypeptide includes at least one post-translational modification. In some embodiments, the at least one post-translational modification comprises attachment of a molecule including but not limited to, a therapeutic enhancing moiety, a label, a dye, a polymer, a water-soluble polymer, a photocrosslinker, a radionuclide, a cytotoxic compound, a drug, an affinity label, a photoaffinity label, a reactive compound, a resin, a second protein or polypeptide or polypeptide analog, an antibody or antibody fragment, a metal chelator, a cofactor, a fatty acid, a carbohydrate, a polynucleotide, a DNA, a RNA, an antisense polynucleotide, a saccharide, a water-soluble dendrimer, a cyclodextrin, an inhibitory ribonucleic acid, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, a quantum dot, a nanotransmitter, a radionucleotide, a radiotransmitter, a neutron-capture agent, or any combination of the above or any other desirable compound or substance, comprising a second reactive group to at least one amino acid comprising a first reactive group utilizing chemistry methodology that is known to one of ordinary skill in the art to be suitable for the particular reactive groups. In certain embodiments, the post-translational modification is made in vivo in a eukaryotic cell or in a non-eukaryotic cell. A linker, polymer, therapeutic enhancing moiety, or other molecule may attach the molecule to the polypeptide. The molecule may be linked directly to the polypeptide.

In certain embodiments, the modified Serp-1 protein includes at least one post-translational modification that is made in vivo by one host cell, where the post-translational modification is not normally made by another host cell type. In certain embodiments, the modified Serp-1 protein includes at least one post-translational modification that is made in vivo by a eukaryotic cell, where the post-translational modification is not normally made by a non-eukaryotic cell. Examples of post-translational modifications include, but are not limited to, glycosylation, acetylation, acylation, lipid-modification, palmitoylation, palmitate addition, phosphorylation, glycolipid-linkage modification, and the like.

Disclosed herein, in some embodiments, are modified Serp-1 proteins comprising a polypeptide and a therapeutic enhancing moiety. In some embodiments, the polypeptide comprises one or more post-translational modification including but not limited to glycosylation, acetylation, acylation, lipid-modification, palmitoylation, palmitate addition, phosphorylation, or glycolipid-linkage modification of the polypeptide. In one embodiment, the post-translational modification comprises attachment of an oligosaccharide to an asparagine by a GlcNAc-asparagine linkage (including but not limited to, where the oligosaccharide comprises (GlcNAc-Man)2-Man-GlcNAc-GlcNAc, and the like). In another embodiment, the post-translational modification comprises attachment of an oligosaccharide (including but not limited to, Gal-GalNAc or Gal-GlcNAc) to a serine or threonine by a GalNAc-serine, a GalNAc-threonine, a GlcNAc-serine, or a GlcNAc-threonine linkage. In certain embodiments, the polypeptide comprises a secretion or localization sequence, an epitope tag, a FLAG tag, a histidine tag comprising one or more histidine residues (e.g. 6 histidine residues (SEQ ID NO: 2)), a GST fusion, and/or the like. Examples of secretion signal sequences include, but are not limited to, a prokaryotic secretion signal sequence, a eukaryotic secretion signal sequence, a eukaryotic secretion signal sequence 5′-optimized for bacterial expression, a novel secretion signal sequence, pectate lyase secretion signal sequence, Omp A secretion signal sequence, and a phage secretion signal sequence. Examples of secretion signal sequences, include, but are not limited to, STII (prokaryotic), Fd GIII and M13 (phage), Bgl2 (yeast), and the signal sequence bla derived from a transposon. Any such sequence may be modified to provide a desired result with the polypeptide, including but not limited to, substituting one signal sequence with a different signal sequence, or substituting a leader sequence with a different leader sequence.

Amino acid side chains of the polypeptide of the modified Serp-1 protein can be modified by utilizing chemistry methodologies known to those of ordinary skill in the art to be suitable for the particular functional groups or substituents. Known chemistry methodologies of a wide variety are suitable for use in this disclosure to incorporate a therapeutic enhancing moiety into the Serp-1 protein. Such methodologies include but are not limited to a Huisgen [3+2] cycloaddition reaction (see, e.g., Padwa, A. in Comprehensive Organic Synthesis, Vol. 4, (1991) Ed. Trost, B. M., Pergamon, Oxford, p. 1069-1109; and, Huisgen, R. in 1,3-Dipolar Cycloaddition Chemistry, (1984) Ed. Padwa, A., Wiley, New York, p. 1-176) with, including but not limited to, acetylene or azide derivatives, respectively.

Some embodiments include conjugates of substances having a wide variety of functional groups, substituents or moieties, with other substances including but not limited to a therapeutic enhancing moiety; a label; a dye; a polymer; a water-soluble polymer; a photocrosslinker; a radionuclide; a cytotoxic compound; a drug; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide; a water-soluble dendrimer; a cyclodextrin; an inhibitory ribonucleic acid; a biomaterial; a nanoparticle; a spin label; a fluorophore, a metal-containing moiety; a radioactive moiety; a novel functional group; a group that covalently or noncovalently interacts with other molecules; a photocaged moiety; an actinic radiation excitable moiety; a photoisomerizable moiety; biotin; a derivative of biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; a quantum dot; a nanotransmitter; a radionucleotide; a radiotransmitter; a neutron-capture agent; or any combination of the above, or any other desirable compound or substance. Some embodiments include conjugates of substances having azide or acetylene moieties with therapeutic enhancing moiety derivatives having the corresponding acetylene or azide moieties. For example, a therapeutic enhancing moiety containing an azide moiety can be coupled to a biologically active molecule at a position in the protein that contains a non-genetically encoded amino acid bearing an acetylene functionality.

As described herein, the present disclosure provides Serp-1 polypeptides coupled to another molecule having the formula Serp-1-L-M, wherein L is a linking group or a chemical bond, and M is any other molecule. In some embodiments, L is stable in vivo or in vitro. In some embodiments, L is hydrolyzable in vivo. In some embodiments, L is metastable in vivo or in vitro

Chemical conjugation can occur by reacting a nucleophilic reactive group of one compound to an electrophilic reactive group of another compound. In some embodiments when L is a bond, the Serp-1 polypeptide is conjugated to M either by reacting a nucleophilic reactive moiety on the Serp-1 polypeptide with an electrophilic reactive moiety on L, or by reacting an electrophilic reactive moiety on the Serp-1 polypeptide with a nucleophilic reactive moiety on M. In embodiments when L is a group that links the Serp-1 polypeptide and M together, the Serp-1 polypeptide and/or M can be conjugated to L either by reacting a nucleophilic reactive moiety on the Serp-1 polypeptide and/or M with an electrophilic reactive moiety on L, or by reacting an electrophilic reactive moiety on the Serp-1 polypeptide and/or M with a nucleophilic reactive moiety on L. Nonlimiting examples of nucleophilic reactive groups include amino, thiol, and hydroxyl. Nonlimiting examples of electrophilic reactive groups include carboxyl, acyl chloride, anhydride, ester, succinimide ester, alkyl halide, sulfonate ester, maleimido, haloacetyl, and isocyanate. In embodiments where the Serp-1 polypeptide and M are conjugated together by reacting a carboxylic acid with an amine, an activating agent can be used to form an activated ester of the carboxylic acid.

Disclosed herein, in some embodiments, are modified Serp-1 proteins comprising a therapeutic enhancing moiety. In some embodiments, the therapeutic enhancing moiety comprises at least one additional Serp-1 protein or modified Serp-1 protein. In some embodiments, the therapeutic enhancing moiety is linked to multiple Serp-1 proteins. In some embodiments, the Serp-1 protein is cross-linked with multiple Serp-1 proteins.

Disclosed herein, in some embodiments, are modified Serp-1 proteins comprising a polypeptide and a therapeutic enhancing moiety. In some embodiments, the therapeutic enhancing moiety is conjugated to a naturally occurring or non-naturally occurring amino acid of the polypeptide. In some embodiments, the therapeutic enhancing moiety is conjugated to a naturally occurring amino acid of the polypeptide. In some embodiments, the therapeutic enhancing moiety is conjugated to a non-naturally occurring amino acid of the polypeptide.

Disclosed herein, in some embodiments, are modified Serp-1 proteins comprising a polypeptide and a therapeutic enhancing moiety. In some embodiments, the therapeutic enhancing moiety is linked to a lysine of the polypeptide. In some embodiments, the therapeutic enhancing moiety is linked to a cysteine of the polypeptide. In some embodiments, the therapeutic enhancing moiety is linked to a cysteine of the polypeptide. In some embodiments, the therapeutic enhancing moiety is randomly conjugated to the polypeptide. In some embodiments, the therapeutic enhancing moiety is randomly conjugated to a lysine of the polypeptide. In some embodiments, the therapeutic enhancing moiety is randomly conjugated to a cysteine of the polypeptide. In some embodiments, the therapeutic enhancing moiety is conjugated to defined location on the polypeptide. In some embodiments, the therapeutic enhancing moiety is conjugated to defined lysine on the polypeptide. In some embodiments, the therapeutic enhancing moiety is conjugated to defined cysteine on the polypeptide. In some embodiments, the therapeutic enhancing moiety conjugated to one or more amino acids comprises a molecular weight of 10 kDa. In some embodiments, the therapeutic enhancing moiety conjugated to one or more amino acids comprises a molecular weight of 5 kDa, 10 kDa, 20 kDa, 30 kDa, or 40 kDa. In some embodiments, the therapeutic enhancing moiety conjugated to one or more amino acids comprises a 10 kDa water soluble polymer. In some embodiments, the therapeutic enhancing moiety conjugated to one or more amino acids comprises a 5 kDa, 10 kDa, 20 kDa, 30 kDa, or 40 kDa water soluble polymer. In some embodiments, the therapeutic enhancing moiety conjugated to one or more lysines comprises a molecular weight of 10 kDa. In some embodiments, the therapeutic enhancing moiety conjugated to one or more lysines comprises a molecular weight of 5 kDa, 10 kDa, 20 kDa, 30 kDa, or 40 kDa. In some embodiments, the therapeutic enhancing moiety conjugated to one or more lysines comprises a 10 kDa water soluble polymer. In some embodiments, the therapeutic enhancing moiety conjugated to one or more lysines comprises a 5 kDa, 10 kDa, 20 kDa, 30 kDa, or 40 kDa water soluble polymer.

Disclosed herein, in some embodiments, are modified Serp-1 proteins comprising a polypeptide and a therapeutic enhancing moiety. In some embodiments, the therapeutic enhancing moiety is chemically conjugated to a site at or near an N-terminus or C-terminus of the polypeptide. In some embodiments, the therapeutic enhancing moiety is chemically conjugated to a site near an N-terminus of the polypeptide. In some embodiments, the therapeutic enhancing moiety is chemically conjugated to a site near a C-terminus of the polypeptide. In some embodiments, the therapeutic enhancing moiety is linked to an end of the polypeptide. In some embodiments, the therapeutic enhancing moiety is linked to an amino terminus of the polypeptide. In some embodiments, the therapeutic enhancing moiety is linked to a carboxyl terminus of the polypeptide. In some embodiments, the therapeutic enhancing moiety at the terminus comprises a molecular weight of 5 kDa. In some embodiments, the therapeutic enhancing moiety at the terminus comprises a molecular weight of 5 kDa, 10 kDa, 20 kDa, 30 kDa, or 40 kDa. In some embodiments, the therapeutic enhancing moiety at the terminus comprises a 5 kDa water soluble polymer. In some embodiments, the therapeutic enhancing moiety at the terminus comprises a 5 kDa, 10 kDa, 20 kDa, 30 kDa, or 40 kDa water soluble polymer.

Disclosed herein, in some embodiments, are modified Serp-1 proteins comprising a polypeptide and a therapeutic enhancing moiety. In some embodiments, the therapeutic enhancing moiety is covalently connected to the polypeptide. In some embodiments, the therapeutic enhancing moiety is connected to the polypeptide through a linker. In some embodiments, the connection through the linker is covalent. Some embodiments include a covalent connection from the therapeutic enhancing moiety to the linker. Some embodiments include a covalent connection from the linker to the polypeptide.

Disclosed herein, in some embodiments, are modified Serp-1 proteins comprising a polypeptide and a therapeutic enhancing moiety. In some embodiments, the at least one therapeutic enhancing moiety comprises 1, 2, 3, 4, 5, 6, 7, 8, 9, 10, 11, 12, 13, 14, 15, 16, 17, 18, 19, 20, 21, 22, 23, 24, or 25 therapeutic enhancing moieties. In some embodiments, the at least one therapeutic enhancing moiety comprises 1, 2, 3, 4, 5, 6, 7, 8, 9, 10, 11, 12, 13, 14, 15, 16, 17, 18, 19, 20, 21, 22, 23, 24, 25, or more therapeutic enhancing moieties, or a range of therapeutic enhancing moieties defined by any two of the aforementioned integers.

In some embodiments, the at least one therapeutic enhancing moiety comprises at least 1 therapeutic enhancing moiety, at least 2 therapeutic enhancing moieties, at least 3 therapeutic enhancing moieties, at least 4 therapeutic enhancing moieties, at least 5 therapeutic enhancing moieties, at least 6 therapeutic enhancing moieties, at least 7 therapeutic enhancing moieties, at least 8 therapeutic enhancing moieties, at least 9 therapeutic enhancing moieties, at least 10 therapeutic enhancing moieties, at least 11 therapeutic enhancing moieties, at least 12 therapeutic enhancing moieties, at least 13 therapeutic enhancing moieties, at least 14 therapeutic enhancing moieties, at least 15 therapeutic enhancing moieties, at least 16 therapeutic enhancing moieties, at least 17 therapeutic enhancing moieties, at least 18 therapeutic enhancing moieties, at least 19 therapeutic enhancing moieties, at least 20 therapeutic enhancing moieties, at least 21 therapeutic enhancing moieties, at least 22 therapeutic enhancing moieties, at least 23 therapeutic enhancing moieties, at least 24 therapeutic enhancing moieties, or at least 25 therapeutic enhancing moieties.

In some embodiments, the no more than one therapeutic enhancing moiety comprises no more than 1 therapeutic enhancing moiety, no more than 2 therapeutic enhancing moieties, no more than 3 therapeutic enhancing moieties, no more than 4 therapeutic enhancing moieties, no more than 5 therapeutic enhancing moieties, no more than 6 therapeutic enhancing moieties, no more than 7 therapeutic enhancing moieties, no more than 8 therapeutic enhancing moieties, no more than 9 therapeutic enhancing moieties, no more than 10 therapeutic enhancing moieties, no more than 11 therapeutic enhancing moieties, no more than 12 therapeutic enhancing moieties, no more than 13 therapeutic enhancing moieties, no more than 14 therapeutic enhancing moieties, no more than 15 therapeutic enhancing moieties, no more than 16 therapeutic enhancing moieties, no more than 17 therapeutic enhancing moieties, no more than 18 therapeutic enhancing moieties, no more than 19 therapeutic enhancing moieties, no more than 20 therapeutic enhancing moieties, no more than 21 therapeutic enhancing moieties, no more than 22 therapeutic enhancing moieties, no more than 23 therapeutic enhancing moieties, no more than 24 therapeutic enhancing moieties, or no more than 25 therapeutic enhancing moieties.

Disclosed herein, in some embodiments, are modified Serp-1 proteins comprising a polypeptide and a therapeutic enhancing moiety. Some embodiments include multiple therapeutic enhancing moieties. In some embodiments, the therapeutic enhancing moieties are randomly conjugated to the polypeptide. In some embodiments, the therapeutic enhancing moieties are conjugated to defined locations on the polypeptide.

Disclosed herein, in some embodiments, are modified Serp-1 proteins comprising a polypeptide and at least one therapeutic enhancing moiety. In some embodiments, the at least one therapeutic enhancing moiety comprises or consists of 5 therapeutic enhancing moieties. In some embodiments, the 5 therapeutic enhancing moieties are randomly conjugated to the polypeptide. In some embodiments, the 5 therapeutic enhancing moieties are conjugated to lysines of the polypeptide.

Disclosed herein, in some embodiments, are modified Serp-1 proteins comprising a polypeptide and at least one therapeutic enhancing moiety. In some embodiments, the therapeutic enhancing moiety includes a moiety (e.g. a pharmacokinetic enhancing moiety, or PKEM) disclosed in PCT publication no. WO2020041636, which is incorporated herein by reference in its entirety.

Disclosed herein, in some embodiments, are modified Serp-1 proteins comprising a polypeptide. In some embodiments, the polypeptide is produced by a cell. In some embodiments, the polypeptide is secreted from the cell. In some embodiments, the cell is a prokaryotic cell. In some embodiments, the cell is a eukaryotic cell. In some embodiments, the eukaryotic cell is a mammalian cell. In some embodiments, the cell comprises a cell line. In some embodiments, the cell line comprises a CHO cell. In some embodiments, the cell comprises a human cell. In some embodiments, the modified Serp-1 protein is purified from the cell or from cell media.

Disclosed herein, in some embodiments, are modified Serp-1 proteins. In some embodiments, the modified Serp-1 protein is purified or is substantially pure. In some embodiments, the modified Serp-1 protein is purified. In some embodiments, the modified Serp-1 protein is substantially pure. In some embodiments, the modified Serp-1 protein is purified from a cell or from cell media. In some embodiments, the modified Serp-1 protein is purified from a cell. In some embodiments, the modified Serp-1 protein is purified from cell media. In some embodiments, the cell media includes a conditioned medium.

Disclosed herein, in some embodiments, are modified Serp-1 proteins. In some embodiments, the modified Serp-1 protein exhibits an in vivo half-life that is greater than a wild-type Serp-1 protein. In some embodiments, the modified Serp-1 protein exhibits an in vivo half-life that is greater than an unmodified Serp-1 protein. In some embodiments, the unmodified Serp-1 protein comprises the polypeptide. In some embodiments, the unmodified Serp-1 protein exhibits an in vivo half-life of at least 1 minute, at least 2 minutes, at least 3 minutes, at least 4 minutes, at least 5 minutes, at least 10 minutes, at least 15 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 1 week, at least 2 weeks, at least 3 weeks, at least 4 weeks, or longer. In some embodiments, the unmodified Serp-1 protein comprises the polypeptide. In some embodiments, the unmodified Serp-1 protein exhibits an in vivo half-life of no more than 1 hour, no more than 2 hours, no more than 3 hours, no more than 4 hours, no more than 5 hours, no more than 6 hours, no more than 7 hours, no more than 8 hours, no more than 9 hours, no more than 10 hours, no more than 11 hours, no more than 12 hours, no more than 13 hours, no more than 14 hours, no more than 15 hours, no more than 16 hours, no more than 17 hours, no more than 18 hours, no more than 19 hours, no more than 20 hours, no more than 21 hours, no more than 22 hours, no more than 23 hours, no more than 1 day, no more than 2 days, no more than 3 days, no more than 4 days, no more than 5 days, no more than 6 days, no more than 1 week, no more than 2 weeks, no more than 3 weeks, or no more than 4 weeks. In some embodiments, the unmodified Serp-1 protein exhibits an in vivo half-life of less than 24 hours. In some embodiments, the unmodified Serp-1 protein exhibits an in vivo half-life of less than 30 minutes. In some embodiments, the unmodified Serp-1 protein exhibits an in vivo half-life of less than about 25 minutes. In some embodiments, the unmodified Serp-1 protein exhibits an in vivo half-life of about 20 minutes. In some embodiments, the unmodified Serp-1 protein exhibits an in vivo half-life of greater than about 15 minutes. In some embodiments, the unmodified Serp-1 protein exhibits an in vivo half-life of greater than about 10 minutes. In some embodiments, the unmodified Serp-1 protein exhibits an in vivo half-life of about 3 minutes. In some embodiments, the unmodified Serp-1 protein exhibits an in vivo half-life of 3.2 minutes. In some embodiments, the in vivo half-life of the unmodified Serp-1 protein is measured in mice. In some embodiments, the in vivo half-life of the unmodified Serp-1 protein is measured in a serum sample. In some embodiments, the in vivo half-life of the unmodified Serp-1 protein is measured using a radioactive label.

In some embodiments, the modified Serp-1 protein exhibits an in vivo half-life that is greater than an unmodified Serp-1 protein. The modified Serp-1 protein may have a half-life that is at least 10%, at least 25%, at least 50%, at least 75%, or at least 100% greater than the unmodified Serp-1 protein. The modified Serp-1 protein may have a half-life that is at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, or at least 30-fold, greater than the unmodified Serp-1 protein. The modified Serp-1 protein may have a half-life that is at least at least about 15-fold greater than the unmodified Serp-1 protein. The modified Serp-1 protein may have a half-life that is at least at least about 20-fold greater than the unmodified Serp-1 protein. The modified Serp-1 protein may have a half-life that is at least at least about 25-fold greater than the unmodified Serp-1 protein. The modified Serp-1 protein may have a half-life that is less than 10%, less than 25%, less than 50%, less than 75%, or less than 100% greater than the unmodified Serp-1 protein. The modified Serp-1 protein may have a half-life that is less than 2-fold, less than 3-fold, less than 4-fold, less than 5-fold, less than 6-fold, less than 7-fold, less than 8-fold, less than 9-fold, less than 10-fold, less than 11-fold, less than 12-fold, less than 13-fold, less than 14-fold, less than 15-fold, less than 16-fold, less than 17-fold, less than 18-fold, less than 19-fold, less than 20-fold, less than 21-fold, less than 22-fold, less than 23-fold, less than 24-fold, less than 25-fold, less than 26-fold, less than 27-fold, less than 28-fold, less than 29-fold, or less than 30-fold, greater than the unmodified Serp-1 protein. The modified Serp-1 protein may have a half-life that is less than less than about 15-fold greater than the unmodified Serp-1 protein. The modified Serp-1 protein may have a half-life that is less than less than about 20-fold greater than the unmodified Serp-1 protein. The modified Serp-1 protein may have a half-life that is less than less than about 25-fold greater than the unmodified Serp-1 protein.

In some embodiments, a modified (e.g. PEGylated) Serp-1 protein has a half-life that is at least a 10-fold greater, at least 15-fold greater, or at least 20-fold greater than an unmodified Serp-1 protein. In some embodiments, a modified Serp-1 protein has a half-life that is up to 20-fold greater, up to 25-fold greater, or up to 30-fold greater than an unmodified Serp-1 protein. For example, a modified (e.g. PEGylated) Serp-1 protein may have a 10 to 30-fold greater half-life than an unmodified Serp-1 protein, or a modified Serp-1 protein may have a 15 to 25-fold greater half-life than an unmodified Serp-1 protein.

In some embodiments, the greater half-life is assessed in a subject. In some embodiments, the greater half-life is determined in a mammal. In some embodiments, the greater half-life is determined in a mammal. For example, some embodiments include a modified (e.g. PEGylated) Serp-1 protein that has a greater half-life than an unmodified Serp-1 protein, as determined in a mouse model. The half-life may be measured in blood (e.g. whole blood, serum, or plasma), or may include a circulating half-life measurement.

In some embodiments, the in vivo half-life is determined in a subject comprising an animal, a vertebrate, a mammal, a rodent, a dog, a rabbit, a horse, cattle, a cat, a sheep, a chicken, a pig, a primate, a non-human primate, or a human. In some embodiments, the half-life is measured in a vertebrate. In some embodiments, the half-life is measured in a mammal. In some embodiments, the half-life is measured in a rodent. In some embodiments, the half-life is measured in a dog. In some embodiments, the half-life is measured in a pig. In some embodiments, the half-life is measured in a primate. In some embodiments, the half-life is measured in a non-human primate. In some embodiments, the half-life is measured in a human.

Disclosed herein, in some embodiments, are modified Serp-1 proteins. In some embodiments, the modified Serp-1 protein exhibits a thermal stability that is greater than a wild-type Serp-1 protein. In some embodiments, the modified Serp-1 protein exhibits a thermal stability that is greater than an unmodified Serp-1 protein. In some embodiments, the modified Serp-1 protein exhibits an in vitro thermal stability that is greater than a wild-type Serp-1 protein. In some embodiments, the modified Serp-1 protein exhibits an in vitro thermal stability that is greater than an unmodified Serp-1 protein. In some embodiments, the unmodified Serp-1 protein comprises the polypeptide. In some embodiments, the modified Serp-1 protein is stable at a temperature of 25° C., 30° C., 35° C., 40° C., 45° C., 50° C., 55° C., 60° C., 65° C., 70° C., 75° C., 80° C., 85° C., 90° C., 95° C., 100° C., or more, or a range of temperatures defined by any two of the aforementioned temperatures. In some embodiments, the modified Serp-1 protein is stable at a temperature of at least 25° C., at least 30° C., at least 35° C., at least 40° C., at least 45° C., at least 50° C., at least 55° C., at least 60° C., at least 65° C., at least 70° C., at least 75° C., at least 80° C., at least 85° C., at least 90° C., at least 95° C., or at least 100° C. In some embodiments, the modified Serp-1 protein is stable at a temperature of at least 55° C. In some embodiments, the modified Serp-1 protein is stable at a temperature of at least about 60° C. In some embodiments, the modified Serp-1 protein is stable at a temperature of at least about 65° C. In some embodiments, the modified Serp-1 protein is stable at a temperature of at least about 70° C. In some embodiments, the modified Serp-1 protein is stable at a temperature of at least about 75° C. In some embodiments, the modified Serp-1 protein is stable at a temperature of at least about 80° C. In some embodiments, the modified Serp-1 protein is stable at a temperature no greater than 25° C., no greater than 30° C., no greater than 35° C., no greater than 40° C., no greater than 45° C., no greater than 50° C., no greater than 55° C., no greater than 60° C., no greater than 65° C., no greater than 70° C., no greater than 75° C., no greater than 80° C., no greater than 85° C., no greater than 90° C., no greater than 95° C., or no greater than 100° C. In some embodiments, the modified Serp-1 protein is stable at a temperature of no greater than about 100° C. In some embodiments, the modified Serp-1 protein is stable at a temperature of no greater than about 125° C. In some embodiments, the modified Serp-1 protein is stable at a temperature of no greater than about 150° C. In some embodiments, the thermal stability comprises maintenance of a biological activity such as u-plasminogen activator (uPA) binding.

Disclosed herein, in some embodiments, are modified Serp-1 proteins exhibiting an in vitro thermal stability that is greater than an unmodified Serp-1 protein. In some embodiments, the unmodified Serp-1 protein is stable at a temperature of 25° C., 30° C., 35° C., 40° C., 45° C., 50° C., 55° C., 60° C., or 65° C., or a range of temperatures defined by any two of the aforementioned temperatures. In some embodiments, the unmodified Serp-1 protein is stable at a temperature of at least 25° C., at least 30° C., at least 35° C., at least 40° C., at least 45° C., at least 50° C., at least 55° C., at least 60° C., or at least 65° C. In some embodiments, the unmodified Serp-1 protein is stable at a temperature of about 25° C. In some embodiments, the unmodified Serp-1 protein is stable at a temperature of about 45° C. In some embodiments, the unmodified Serp-1 protein is stable at a temperature of about 55° C. In some embodiments, the unmodified Serp-1 protein is stable at a temperature of about 65° C. In some embodiments, the unmodified Serp-1 protein is stable at a temperature no greater than 25° C., no greater than 30° C., no greater than 35° C., no greater than 40° C., no greater than 45° C., no greater than 50° C., no greater than 55° C., no greater than 60° C., or no greater than 65° C. In some embodiments, the unmodified Serp-1 protein is stable at a temperature of no greater than about 70° C. In some embodiments, the unmodified Serp-1 protein is stable at a temperature of no greater than about 65° C. In some embodiments, the unmodified Serp-1 protein is stable at a temperature of no greater than about 60° C. In some embodiments, the unmodified Serp-1 protein is stable at a temperature of no greater than about 55° C. In some embodiments, the unmodified Serp-1 protein is unstable at a temperature of about 60° C. In some embodiments, the unmodified Serp-1 protein is unstable at a temperature of about 65° C. In some embodiments, the unmodified Serp-1 protein is unstable at a temperature of about 75° C. In some embodiments, the unmodified Serp-1 protein is unstable at a temperature of about 85° C. In some embodiments, the unmodified Serp-1 protein is unstable at a temperature of about 95° C. In some embodiments, the unmodified Serp-1 protein is unstable at a temperature of about 60° C. or higher. In some embodiments, the unmodified Serp-1 protein is unstable at a temperature of about 65° C. or higher. In some embodiments, the unmodified Serp-1 protein is unstable at a temperature of about 75° C. or higher. In some embodiments, the unmodified Serp-1 protein is unstable at a temperature of about 85° C. or higher. In some embodiments, the unmodified Serp-1 protein is unstable at a temperature of about 95° C. or higher.

Disclosed herein, in some embodiments, are modified Serp-1 proteins exhibiting an in vitro thermal stability that is greater than a wild-type or unmodified Serp-1 protein. In some embodiments, the stability is at a temperature for a period of time. In some embodiments, the stability lasts at least 1 minute, at least 2 minutes, at least 3 minutes, at least 4 minutes, at least 5 minutes, at least 10 minutes, at least 15 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 1 week, at least 2 weeks, at least 3 weeks, or at least 4 weeks. In some embodiments, the stability lasts no more than 15 minutes, no more than 30 minutes, no more than 45 minutes, no more than 1 hour, no more than 2 hours, no more than 3 hours, no more than 4 hours, no more than 5 hours, no more than 6 hours, no more than 7 hours, no more than 8 hours, no more than 9 hours, no more than 10 hours, no more than 11 hours, no more than 12 hours, no more than 13 hours, no more than 14 hours, no more than 15 hours, no more than 16 hours, no more than 17 hours, no more than 18 hours, no more than 19 hours, no more than 20 hours, no more than 21 hours, no more than 22 hours, no more than 23 hours, no more than 1 day, no more than 2 days, no more than 3 days, no more than 4 days, no more than 5 days, no more than 6 days, no more than 1 week, no more than 2 weeks, no more than 3 weeks, or no more than 4 weeks. In some embodiments, the stability lasts about 5 minutes. In some embodiments, the stability lasts for at least about 5 minutes. In some embodiments, the stability lasts about 2 hours. In some embodiments, the stability is at a temperature for about 2 hours. In some embodiments, the stability is at a temperature for about 5 minutes. In some embodiments, the stability is at a temperature for at least about 5 minutes.

In some embodiments, the thermal stability comprises a maintenance of a 3-dimensional conformation or lack of denaturation. In some embodiments, the maintenance of a 3-dimensional conformation or lack of denaturation is determined by an ability of the modified Serp-1 protein to bind to an anti-Serp-1 antibody. In some embodiments, the maintenance of a 3-dimensional conformation or lack of denaturation is determined by an ability of the modified Serp-1 protein to bind to uPA, as indicated by binding of a modified Serp-1-uPA complex to an anti-uPA antibody.

Disclosed herein, in some embodiments, are modified Serp-1 proteins comprising a polypeptide and a therapeutic enhancing moiety. In some embodiments, the biological activity of the modified Serp-1 protein comprises binding to u-plasminogen activator (uPA). In some embodiments, the binding between the modified Serp-1 protein and uPA comprises a binding affinity with an equilibrium dissociation constant (Kd) below 1 mM, below 750 μM, below 500 μM, below 250 μM, below 200 μM, below 150 μM, below 100 μM, below 75 μM, below 50 μM, a Kd below 45 μM, a Kd below 40 μM, a Kd below 35 μM, a Kd below 30 μM, a Kd below 25 μM, a Kd below 20 μM, a Kd below 15 μM, a Kd below 14 μM, a Kd below 13 μM, a Kd below 12 μM, a Kd below 11 μM, a Kd below 10 μM, a Kd below 9 μM, a Kd below 8 μM, a Kd below 7 μM, a Kd below 6 μM, a Kd below 5 μM, a Kd below 4 μM, a Kd below 3 μM, a Kd below 2 μM, or a Kd below 1 μM. In some embodiments, the binding between the modified Serp-1 protein and uPA comprises a binding affinity with an equilibrium dissociation constant (Kd) above 1 mM, above 750 μM, above 500 μM, above 250 μM, above 200 μM, above 150 μM, above 100 μM, above 75 μM, above 50 μM, a Kd above 45 μM, a Kd above 40 μM, a Kd above 35 μM, a Kd above 30 μM, a Kd above 25 μM, a Kd above 20 μM, a Kd above 15 μM, a Kd above 14 μM, a Kd above 13 μM, a Kd above 12 μM, a Kd above 11 μM, a Kd above 10 μM, a Kd above 9 μM, a Kd above 8 μM, a Kd above 7 μM, a Kd above 6 μM, a Kd above 5 μM, a Kd above 4 μM, a Kd above 3 μM, a Kd above 2 μM, or a Kd above 1 μM. In some embodiments, the binding between the modified Serp-1 protein and uPA comprises a binding affinity with an equilibrium dissociation constant (Kd) of about 1 mM, of about 750 μM, of about 500 μM, of about 250 μM, of about 200 μM, of about 150 μM, of about 100 μM, of about 75 μM, of about 50 μM, a Kd of about 45 μM, a Kd of about 40 μM, a Kd of about 35 μM, a Kd of about 30 μM, a Kd of about 25 μM, a Kd of about 20 μM, a Kd of about 15 μM, a Kd of about 14 μM, a Kd of about 13 μM, a Kd of about 12 μM, a Kd of about 11 μM, a Kd of about 10 μM, a Kd of about 9 μM, a Kd of about 8 μM, a Kd of about 7 μM, a Kd of about 6 μM, a Kd of about 5 μM, a Kd of about 4 μM, a Kd of about 3 μM, a Kd of about 2 μM, or a Kd of about 1 μM, or a range of Kd values defined by any two of the aforementioned Kd values.

Disclosed herein, in some embodiments, is a culture medium, or an isolated cell, vector, plasmid, prokaryotic cell, eukaryotic cell, virus, AAV, mammalian cell, yeast, bacterium, or cell-free translation system comprising a modified Serp-1 protein as disclosed herein. Some embodiments include a composition comprising the culture medium, or isolated cell, vector, plasmid, prokaryotic cell, eukaryotic cell, virus, AAV, mammalian cell, yeast, bacterium, or cell-free translation system ad disclosed herein, and a pharmaceutically acceptable carrier. In some embodiments, the pharmaceutically acceptable carrier comprises a buffer. Some embodiments relate to a culture medium comprising a modified Serp-1 protein as disclosed herein. Some embodiments include a composition comprising or consisting of the culture medium.

Disclosed herein, in some embodiments, are compositions comprising the modified Serp-1 protein as described herein. In some embodiments, the composition further comprises a pharmaceutically acceptable carrier. Disclosed herein, in some embodiments, are compositions comprising the modified Serp-1 protein as described herein, and a pharmaceutically acceptable carrier. In some embodiments, the pharmaceutically acceptable carrier comprises a buffer. Some embodiments include one or more other active compounds comprising a drug, a vaccine, an antibiotic, an antiviral compound, or an anti-parasitic compound. In some embodiments, the composition is a pharmaceutical composition. In some embodiments, the composition is sterile.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

Disclosed herein, in some embodiments, are modified Serp-1 proteins or compositions containing a modified Serp-1 protein as described herein, for use as a medicament. Disclosed herein, in some embodiments, are uses of a modified Serp-1 protein or composition containing a modified Serp-1 protein as described herein for the manufacture of a medicament for treating or preventing a disease or disorder.

Disclosed herein, in some embodiments, are expression cassettes comprising a nucleic acid encoding the modified Serp-1 protein as described herein. In some embodiments, the nucleic acid comprises DNA. In some embodiments, the expression cassette is configured for expression in a cell. In some embodiments, the cell comprises a mammalian cell. In some embodiments, the cell is a CHO cell. In some embodiments, the cell is a human cell.

Some embodiments may include the use of routine techniques in the field of recombinant genetics for, for example, cloning, expressing, and purifying a Serp-1 polypeptide or a modified Serp-1 protein. Basic texts disclosing some general methods include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

General texts which describe molecular biological techniques include Berger and Kimmel, Guide to Molecular Cloning Techniques, Methods in Enzymology volume 152 Academic Press, Inc., San Diego, Calif. (Berger); Sambrook et al., Molecular Cloning: A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). These texts describe mutagenesis, the use of vectors, promoters and many other relevant topics related to, including but not limited to, the generation of genes or polynucleotides that include selector codons for production of proteins that include unnatural amino acids, orthogonal tRNAs, orthogonal synthetases, and pairs thereof.

Several well-known methods of introducing target nucleic acids into cells are available, any of which can be used. These include: fusion of the recipient cells with bacterial protoplasts containing the DNA, electroporation, projectile bombardment, and infection with viral vectors (discussed further, below), etc. Bacterial cells can be used to amplify a number of plasmids containing DNA constructs. The bacteria are grown to log phase and the plasmids within the bacteria can be isolated by a variety of methods known in the art (see, for instance, Sambrook). In addition, kits are commercially available for the purification of plasmids from bacteria, (see, e.g., EasyPrep™, FlexiPrep™, both from Pharmacia Biotech; StrataClean™ from Stratagene; and, QIAprep™ from Qiagen). The isolated and purified plasmids may then be further manipulated to produce other plasmids, used to transfect cells or incorporated into related vectors to infect organisms.

Serp-1 polypeptides or modified Serp-1 proteins described herein may purified after expression in recombinant systems. The Serp-1 polypeptide or modified Serp-1 protein may be purified from host cells or culture medium by a variety of methods known to the art. Recombinant host cells may be disrupted or homogenized to release Serp-1 polypetides or modified Serp-1 proteins from within the cells using a variety of methods known to those of ordinary skill in the art. Host cell disruption or homogenization may be performed using well known techniques including, but not limited to, enzymatic cell disruption, sonication, dounce homogenization, or high pressure release disruption.

In the case of a soluble Serp-1 polypeptide or a soluble modified Serp-1 protein, the Serp-1 polypeptide or modified Serp-1 protein may be secreted into a periplasmic space or into a culture medium. In addition, soluble Serp-1 polypeptide or modified Serp-1 protein may be present in the cytoplasm of the host cells. It may be desired to concentrate soluble Serp-1 polypeptide or modified Serp-1 protein prior to performing purification steps. Standard techniques known to those of ordinary skill in the art may be used to concentrate soluble Serp-1 polypeptide or modified Serp-1 protein from, for example, cell lysates or culture medium. In addition, standard techniques known to those of ordinary skill in the art may be used to disrupt host cells and release soluble Serp-1 polypeptide or modified Serp-1 protein from the cytoplasm or periplasmic space of the host cells.

A Serp-1 polypeptide or modified Serp-1 protein described herein may also be purified to remove DNA from the protein solution. DNA may be removed by any suitable method known to the art, such as precipitation or ion exchange chromatography, but may be removed by precipitation with a nucleic acid precipitating agent, such as, but not limited to, protamine sulfate. The Serp-1 polypeptide or modified Serp-1 protein may be separated from the precipitated DNA using standard methods.

Any of the following exemplary procedures can be employed for purification of a Serp-1 polypeptide or modified Serp-1 protein: affinity chromatography; anion- or cation-exchange chromatography (using, including but not limited to, DEAE SEPHAROSE); chromatography on silica; high performance liquid chromatography (HPLC); reverse phase HPLC; gel filtration (using, including but not limited to, SEPHADEX G-75); hydrophobic interaction chromatography; size-exclusion chromatography; metal-chelate chromatography; ultrafiltration/diafiltration; ethanol precipitation; ammonium sulfate precipitation; chromatofocusing; displacement chromatography; electrophoretic procedures (including but not limited to preparative isoelectric focusing), differential solubility (including but not limited to ammonium sulfate precipitation), SDS-PAGE, or extraction.

### II. METHODS AND USES

Disclosed herein, in some embodiments, are methods. Some embodiments relate to methods of administering a composition described herein to a subject. Some embodiments relate to methods of treatment comprising administering a composition described herein to a subject. Some embodiments relate to use a composition described herein, such as administering the composition to a subject.

Some embodiments relate to a method of treating a disorder in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject. Some embodiments include a method of immune modulation comprising administering a composition described herein to a subject in need thereof.

In some embodiments, the disorder comprises a hemorrhage. In some embodiments, the disorder comprises diffuse alveolar hemorrhage (DAH). In some embodiments, DAH is a rare and potentially fatal complication which manifests in some patients. DAH may have a 50-80% mortality rate. Features of DAH may include vascular dysfunction with capillaritis, hemorrhage, interstitial infiltration of inflammatory cells, tissue necrosis and/or deposition of hemosiderin-laden macrophages. The therapeutic options for management and treatment of DAH have been limited, with up to 98% of patients receiving elevated doses corticosteroids, cyclophosphamide or other immune suppressants. Despite a small number of case reports describing success with these treatments, there persists a high overall mortality rate in DAH. Paradoxically, high doses of corticosteroids have also been associated with the onset of DAH and pose the additional risk of infection, which is a critical comorbidity for patients with DAH and results in a poor prognosis. Recent efforts to find new approaches for mitigating the devastating outcomes of DAH include off-label use of recombinant clotting factor VII (rFVIIa) delivered by a bronchoscope with the potential for development of harmful thrombosis, or the initiation of extracorporeal membrane oxygenation (ECMO) support with the potential for increased alveolar bleeding due to the requirement for simultaneous intravenous heparin. Thus, there remains an unmet need for safe and effective steroid-sparing or -replacing treatments for patients with DAH. The modified Serp-1 proteins described herein may meet this need.

While the initiating events of DAH in patients remains unclear, specific components of the molecular mechanism have been elucidated in experimental settings. The pristane-induced model of DAH in C57BL6/J mice is an experimental system for studying associated DAH and recapitulates components of the human disease including capillaritis, hemorrhage and interstitial infiltration of inflammatory cells, tissue necrosis and deposition of hemosiderin-laden macrophages. A clinical association in some patients presenting with DAH is a low level of circulating complement C3. In the pristane model, deposition of complement C3 in the pulmonary microvasculature initiates inflammatory cell infiltration leading to capillaritis and vascular permeability and the development of alveolar hemorrhage. Mice with a knockout of C3 or CD18, a component of the C3 receptor highly expressed in pulmonary macrophages and subsets of pneumocytes, are resistant to pristane-induced lung pathology and demonstrate that a functional complement response is a necessary requirement for the onset of DAH. Impaired macrophage-mediated clearance of apoptotic cells is strongly associated with human DAH and a critical role has been defined for macrophages in the development of pristane-induced DAH. Systemic depletion of macrophages by chlodronate liposome or pharmacologic modulation of macrophage polarity towards a pro-resolution M2 phenotype potently suppresses the development of DAH pathology. M2-polarized alveolar macrophages are an IL-10-producing and apoptotic cell-clearing cell type in the lung and knockout of IL-10 worsens the severity of DAH in pristane-treated mice. Thus, one or more pathways responsible for macrophage-mediated resolution of vascular dysfunction in the lungs may be a potential target for developing novel treatments for DAH. Serp-1 may modulate multiple aspects of pathways known to be important for pathogenesis of DAH in a protective manner (see, e.g., FIG. 2A-2C). In some embodiments, the administration improves an aspect of DAH as provided in Example 3.

In some embodiments the compositions described herein are administered to treat lung consolidation or hemorrhage in severe viral infections and sepsis (e.g. acute respiratory distress syndrome, an Ebola virus infection, or a coronavirus infection such as coronavirus disease 2019), or in inflammatory vascular syndromes from medium to large artery disease (e.g. giant cell arteritis or Takayasu's), transplant rejection and vasculitis, inflammatory vascular disease in inflammatory bowel disease (e.g. ulcerative colitis or Crohn's disease), systemic autoimmune rheumatological disorders (e.g. rheumatoid arthritis, psoriatic arthritis, or systemic lupus erythematosus), or unstable atherosclerosis causing heart attack and stroke or peripheral vascular disease. The composition may be used to treat a subject having lupus. The composition may be used to treat DAH in a subject having lupus.

Some embodiments include administering the composition described herein to a subject. In some embodiments, the subject an animal, a vertebrate, a mammal, a rodent, a dog, a rabbit, a horse, cattle, a cat, a sheep, a chicken, a pig, a primate, or a non-human primate. In some embodiments, the subject is a mammal. In some embodiments, the subject is a human.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

It is to be understood that this disclosure is not limited to the particular methodology, protocols, cell lines, constructs, and reagents described herein and as such may vary. It is also to be understood that the terminology used herein is for the purpose of describing particular embodiments only, and is not intended to limit the scope of the present disclosure, which will be limited only by the appended claims.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a” “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

A “non-naturally encoded amino acid” may include an amino acid that is not one of the 20 common amino acids or pyrrolysine or selenocysteine. Other terms that may be used synonymously with the term “non-naturally encoded amino acid” are “non-natural amino acid,” “unnatural amino acid,” “non-naturally-occurring amino acid,” and variously hyphenated and non-hyphenated versions thereof. The term “non-naturally encoded amino acid” also includes, but is not limited to, amino acids that occur by modification (e.g. post-translational modifications) of a naturally encoded amino acid (including but not limited to, the 20 common amino acids or pyrrolysine and selenocysteine) but are not themselves naturally incorporated into a growing polypeptide chain by the translation complex. Examples of such non-naturally-occurring amino acids include, but are not limited to, para-acetylphenylalanine, N-acetylglucosaminyl-L-serine, N-acetylglucosaminyl-L-threonine, and O-phosphotyrosine.

The term “substantially pure” may refer to a Serp-1 polypeptide or modified Serp-1 protein that may be substantially or essentially free of components that normally accompany or interact with the protein as found in its naturally occurring environment, i.e. a native cell, or host cell in the case of recombinantly produced Serp-1 polypeptide or modified Serp-1 protein. Serp-1 polypeptide or modified Serp-1 protein that may be substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. “Substantially purified” Serp-1 polypeptide or modified Serp-1 protein as produced by methods described herein may have a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

The term “isolated,” when applied to a nucleic acid or protein, may denote that the nucleic acid or protein is free of at least some of the cellular components with which it is associated in the natural state, or that the nucleic acid or protein has been concentrated to a level greater than the concentration of its in vivo or in vitro production. It can be in a homogeneous state. Isolated substances can be in either a dry or semi-dry state, or in solution, including but not limited to, an aqueous solution. It can be a component of a pharmaceutical composition that comprises additional pharmaceutically acceptable carriers and/or excipients. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to substantially one band in an electrophoretic gel. Particularly, it may mean that the nucleic acid or protein is at least 85% pure, at least 90% pure, at least 95% pure, at least 99% or greater pure.

A “recombinant host cell” or “host cell” may refer to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “medium” or “media” may include any culture medium, solution, solid, semi-solid, or rigid support that may support or contain any host cell, including bacterial host cells, yeast host cells, insect host cells, plant host cells, eukaryotic host cells, mammalian host cells, CHO cells, prokaryotic host cells, E. coli, or Pseudomonas host cells, and/or cell contents. Thus, the term may encompass a medium in which the host cell has been grown, e.g., a medium into which a Serp-1 polypeptide or modified Serp-1 protein has been secreted, including a medium either before or after a proliferation step. The term also may encompass buffers or reagents that contain host cell lysates, such as in the case where a Serp-1 polypeptide or modified Serp-1 protein is produced intracellularly and the host cells are lysed or disrupted to release the Serp-1 polypeptide or modified Serp-1 protein.

The term “amino acid” may refer to naturally occurring and/or non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs may include compounds that have the same basic chemical structure as a naturally occurring amino acid, i.e., an a carbon that is bound to a hydrogen, a carboxyl group, an amino group, and an R group, such as homoserine, norleucine, methionine sulfoxide, methionine methyl sulfonium. Such analogs may have modified R groups (such as norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, para-acetylphenylalanine, α-methyl amino acids (e.g., α-methyl alanine), D-amino acids, histidine-like amino acids (e.g., 2-amino-histidine, β-hydroxy-histidine, homohistidine, α-fluoromethyl-histidine and α-methyl-histidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the proteins of the present disclosure may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

The term, “functional group”, “active moiety”, “activating group”, “leaving group”, “reactive site”, “chemically reactive group” or “chemically reactive moiety” may be used to refer to distinct, definable portions or units of a molecule. The terms may be somewhat synonymous in the chemical arts and be used herein to indicate the portions of molecules that perform some function or activity and are reactive with other molecules.

The term “linkage” or “linker” may include a group or bond formed as a result of a chemical reaction, and include a covalent linkage. Linkers may include but are not limited to short linear, branched, multi-armed, or dendrimeric molecules such as polymers.

A “biologically active molecule”, “biologically active moiety” or “biologically active agent” may include a substance which can affect any physical or biochemical properties of a biological system, pathway, molecule, or interaction relating to an organism, including but not limited to, viruses, bacteria, bacteriophage, transposon, prion, insects, fungi, plants, animals, and humans. In particular, a biologically active molecule may include, but is not limited to, a substance intended for diagnosis, cure, mitigation, treatment, or prevention of disease in humans or other animals, or to otherwise enhance physical or mental well-being of humans or animals. Examples of biologically active molecules may include, but are not limited to, peptides, proteins, polymers, enzymes, small molecule drugs, vaccines, immunogens, hard drugs, soft drugs, carbohydrates, inorganic atoms or molecules, dyes, lipids, nucleosides, radionuclides, oligonucleotides, toxoids, toxins, prokaryotic and eukaryotic cells, viruses, polysaccharides, nucleic acids and portions thereof obtained or derived from viruses, bacteria, insects, animals or any other cell or cell type, liposomes, microparticles, or micelles. Classes of biologically active agents that may be suitable for use herein include, but are not limited to, drugs, prodrugs, radionuclides, imaging agents, polymers, antibiotics, fungicides, anti-viral agents, anti-inflammatory agents, immune modulating agents, anti-tumor agents, cardiovascular agents, anti-anxiety agents, hormones, growth factors, steroidal agents, microbially derived toxins, and the like.

The terms “electrophilic group”, “electrophile” and the like may refer to an atom or group of atoms that can accept an electron pair to form a covalent bond. An “electrophilic group” may include but is not limited to a halide, carbonyl and epoxide containing compound. Common electrophiles may be halides such as thiophosgene, glycerin dichlorohydrin, phthaloyl chloride, succinyl chloride, chloroacetyl chloride, chlorosucciriyl chloride, etc.; ketones such as chloroacctone, bromoacetone, etc.; aldehydes such as glyoxal, etc.; isocyanates such as hexamethylene diisocyanate, tolylene diisocyanate, meta-xylylene diisocyanate, cyclohexylmethane-4,4-diisocyanate, etc and derivatives of these compounds may be used.

The terms “nucleophilic group”, “nucleophile” and the like may refer to an atom or group of atoms that have an electron pair capable of forming a covalent bond. Groups of this type may be iohizable groups that react as anionic groups. A “nucleophilic group” may include but is not limited to any of hydroxyl, primary amines, secondary amines, tertiary amines and thiols.

The terms “identical” or percent “identity,” in the context of two or more nucleic acids or polypeptide sequences, may refer to two or more sequences or subsequences that are the same. Sequences are “substantially identical” if they have a percentage of amino acid residues or nucleotides that are the same (i.e., about 60% sequence identity, about 65%, about 70%, about 75%, about 80%, about 85%, about 90%, or about 95% sequence identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition may also refer to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide.

### VI. EXAMPLES

### Example 1: Prior Therapeutic Development Activities of Unmodified Serp-1 Proteins

Wildtype, unmodified Serp-1 has previously been studied for use in heart disease in clinical settings. Serp-1 was studied in patients with acute coronary syndrome undergoing stent implant. These studies on Serp-1 in heart disease patients provided information relevant to the safety and pharmacokinetics of wildtype, unmodified Serp-1 protein in vivo.

A previous 28-day intravenous bolus GLP toxicity study using wildtype, unmodified Serp-1 protein in albino rats with a 14-day recovery period at doses of 0, 0.07, 0.5 and 3.5 mg/kg/day indicated no effect on ophthalmology, urinalysis, clinical chemistry or food consumption. A transient, dose-dependent effect on muscle tone, activity and/or respiration rate was observed within minutes of dosing between days 9 and 14 but resolved within two hours. At the highest doses, clinical signs of potential neutralizing antibody development were observed as well as a nominal decrease in body weight in the fourth week of dosing. A decrease in monocyte activation was observed at the highest dose, but no effect was observed on NK activity or distribution of lymphocyte subsets. All changes reversed in the recovery period.

A previous 14-day intravenous bolus GLP toxicity study using wildtype, unmodified Serp-1 protein in cynomolgus monkeys with a 14-day recovery period at doses of 0, 0.03, 0.15 and 0.75 mg/kg/day indicated no effects on body weight, hematology or clinical chemistry. Histological assessment of adrenals, bone, bone marrow, heart, injection site, kidney, liver, lungs, lymph nodes, spleen and thymus indicated no effect. No effect was observed on NK cell activity, monocyte activation or in distributions of lymphocyte subsets. Non-neutralizing anti-drug antibodies were detected.

The circulating half-life of wildtype, unmodified Serp-1 protein Serp-1 was previously extended from approximately 30 minutes up to approximately 2 hours with passive glycoengineering by transitioning protein production from a hollow fiber bioreactor to a stirred tank bioreactor, resulting in an increase in the sialylation of Serp-1 with Neu5Ac (increased from 0.2 to 6.2). Dose-range finding studies identified a no-observed-adverse-effect-level (NOAEL) of 12.5 mg/kg in rats and 6.0 mg/kg in monkeys. Thus, the pharmacokinetic properties of Serp-1 can be modulated to increase half-life.

### Example 2: Biological Activity and Enhanced Properties of Modified Serp-1 Proteins

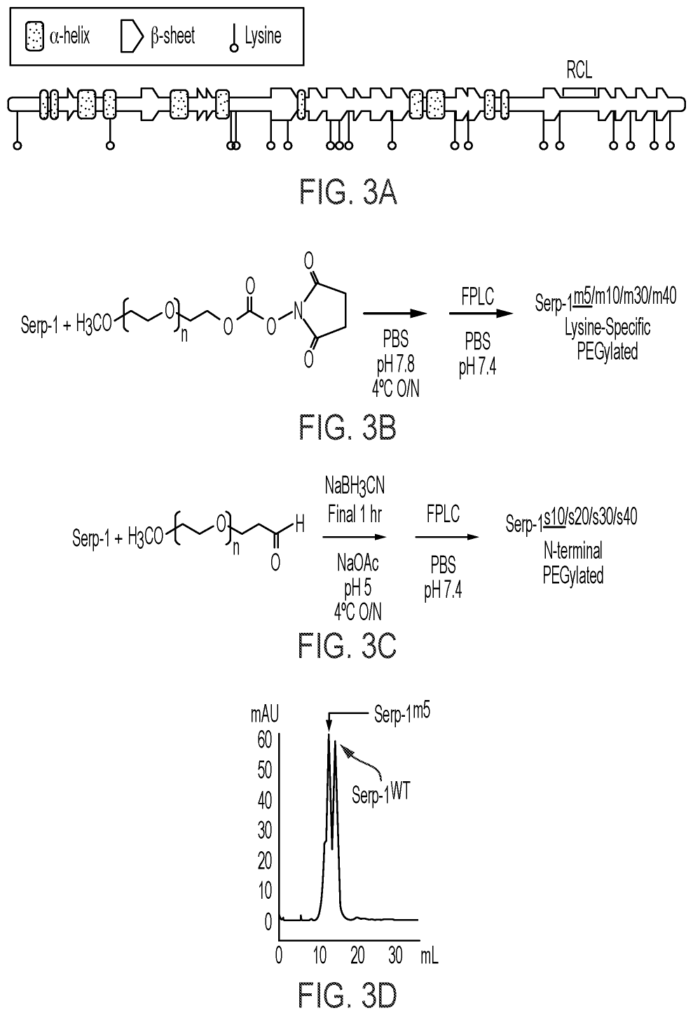

Modified Serp-1 proteins were made, including a Serp-1 polypeptide and therapeutic enhancing moieties each comprising a water soluble polymer comprising polyethylene glycol (PEG). A laboratory-scale small batch reaction was performed to produce a PEGylated Serp-1 (see FIG. 3A-3D). Multiple approaches were made and tested: (1) addition of multiple 5 kDa PEG moieties non-specifically to any amenable lysine residues of Serp-1 (modSerp-1^{m5}, where “m” refers to multi-site), and (2) addition of a 10 kDa PEG moiety to the amino (N)-terminus of Serp-1 (modSerp-1^{s10}, where “s” refers to single-site).

The first modified Serp-1 protein (modSerp-1^{m5}) included therapeutic enhancing moieties comprising PEG randomly conjugated to lysines of the polypeptide. ModSerp-1^{m5} was made using multi-site modification, performed by incubation of Serp-1 with a methoxy-N-hydroxylsuccinimide (NHS) version of the therapeutic enhancing moiety (methoxy-PEG5K-NHS) in phosphate buffered saline (PBS), pH 7.8 overnight at 4° C. No quenching of the NHS reaction was necessary. The reaction products comprising the modSerp-1^{m5} were purified by FPLC over a SuperDex-200 column in PBS, pH 7.4 and preservation of inhibitory function was tested in a reaction with recombinant, active urokinase-type plasminogen activator (uPA).

The second modified Serp-1 protein (modSerp-1^{s10}) included 10 of the therapeutic enhancing moieties conjugated to an amino terminus of the polypeptide. ModSerp-1^{s10} was made using incubation of Serp-1 with a methoxy-propionaldehye version of the therapeutic enhancing moiety (methoxy-PEG10K-propionaldehye) in sodium acetate buffer (NaOAc), pH 5 overnight at 4° C. The reaction was quenched during the last hour of incubation with sodium cyanoborohydride (NaBH3CN).

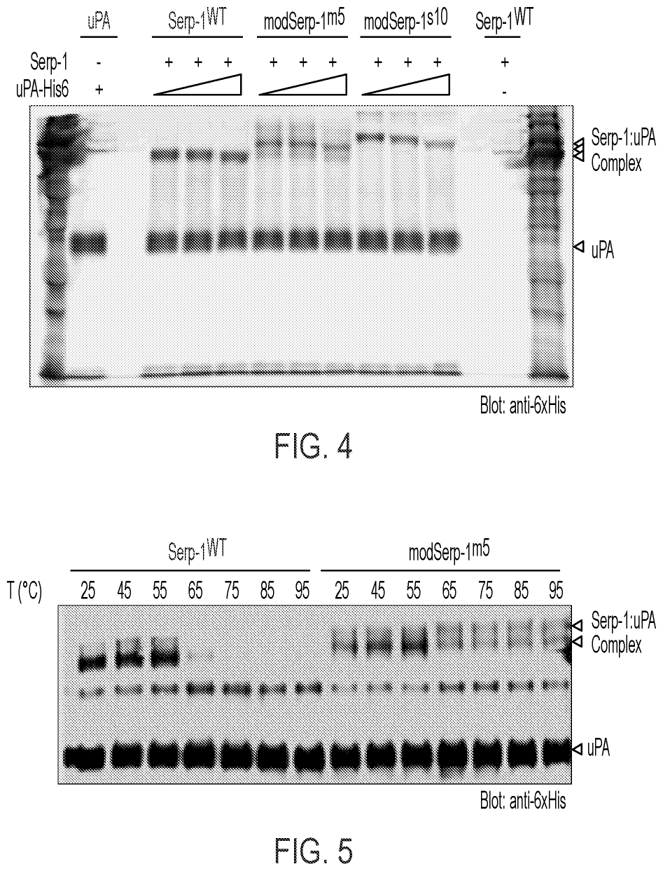

As shown in FIG. 4, both modified Serp-1 proteins bound urokinase-type plasminogen activator (uPA) when combined with uPA, indicating that the modified Serp-1 proteins were biologically active.

As shown in FIG. 5, modSerp-1^{m5} exhibited increased thermal stability compared to a wild-type Serp-1 protein, as determined by an in vitro assay. The assay included incubating the modified or wild-type Serp-1 proteins for 5 minutes at the various temperatures indicated, then cooled with ice, and incubated with an anti-uPA antibody for 2 hours at 25° C.

The results of these experiments indicate at least the following conclusions: that Serp-1 can be PEGylated and purified without the need for renaturation or complex buffer exchanges; and that the biological activity Serp-1 including the inhibitory function is preserved through the PEGylation reaction and purification procedure. Further, PEGylation conferred enhanced thermostability to Serp-1. Likewise, other amounts or types of PEGylation (or other water soluble polymer) would be expected to confer similar enhanced effects on a Serp-1 protein. For example, a modified Serp-1 protein comprising a Serp-1 polypeptide conjugated to one or more therapeutic enhancing moieties comprising PEG molecules at various locations and having any of the following molecular weights are expected to also provide improved properties such as heat stability and/or a biological activity such as uPA binding: 5 kDa, 10 kDa, 15 kDa, 20 kDa, 25 kDa, 30 kDa, 35 kDa, or 40 kDa, or a range of molecular weights (e.g. 5-40 kDa, or a range that includes of any of the aforementioned weights).

### Example 3: In Vivo Biological Activity of Modified Serp-1 Proteins

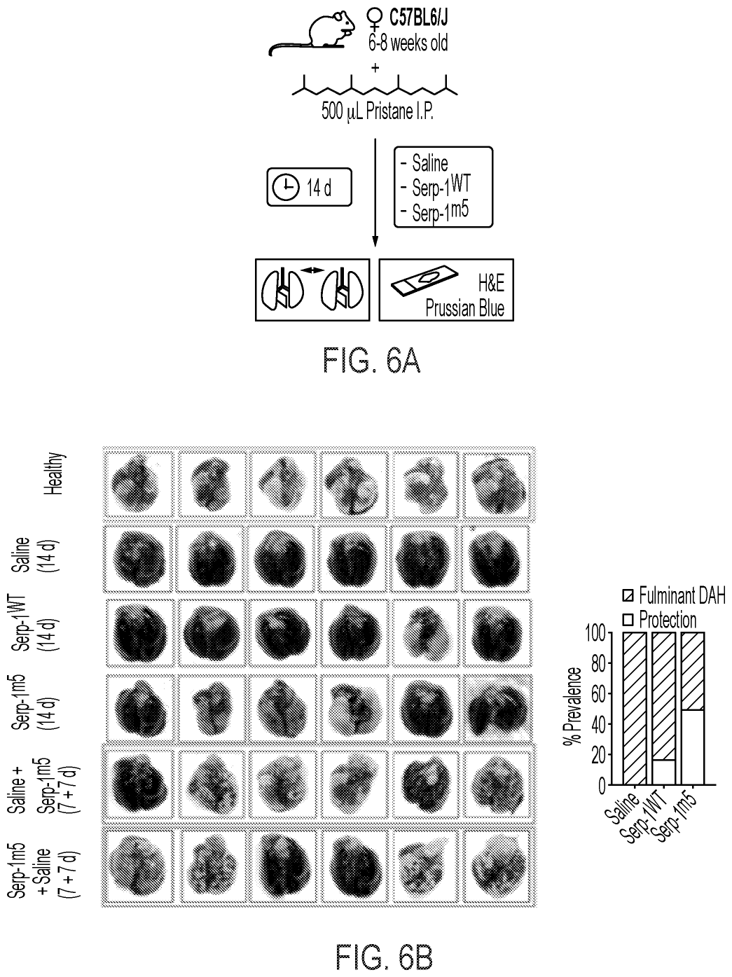

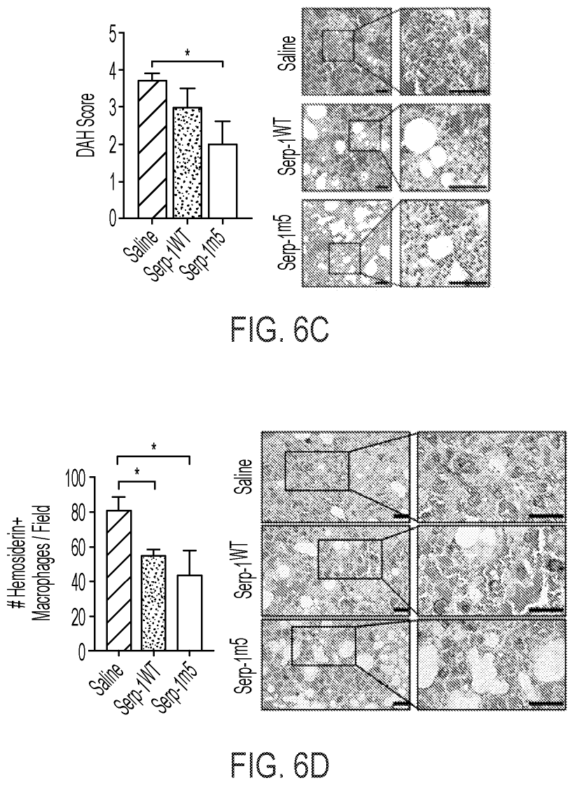

Experimental pristane-induced diffuse alveolar hemorrhage (DAH) was used in 6-week old female C57BL6/J mice (see FIG. 6A-6D). Mice received either saline (N=6), 100 ng/g unmodified Serp-1 (N=6) or 100 ng/g modSerp-1^{m5} (N=6) daily by intraperitoneal injection for 14 days. Gross pathology for pulmonary hemorrhage and histopathology for alveolar hemorrhage and hemosiderin-laden macrophage deposition was performed and T-cell counts in the spleen were performed by flow cytometry. Gross pathology indicated that 6/6 (100%) of saline-treated mice, 5/6 (83%) of wildtype Serp-1-treated mice and only 3/6 (50%) of modSerp-1^{m5}-treated mice exhibited pulmonary hemorrhage. Histopathology and DAH scoring by hematoxylin and eosin (H&E) staining showed a significant (p<0.05) reduction in DAH score in mice treated with modSerp-1^{m5} Both wildtype Serp-1 and modSerp-1^{m5} reduced the deposition of hemosiderin-laden macrophages (p<0.05) by Prussian Blue staining.

A significant reduction in lung hemorrhage was evident when the PEGylated Serp-1 construct is given 7 days after pristane. This suggests that modified Serp-1 (e.g. Serp-1 conjugated to a therapeutic enhancing moiety such as PEG) may be useful in a clinical setting where a patient is admitted after onset of disease and is not limited to prophylactic treatment.

FIG. 6B shows that the modified Serp-1 proteins may improve gross pathology. Particularly, the modified Serp-1 protein substantially ameliorated the frequency of gross pathology in experimental DAH. Delayed modified Serp-1 (PEG-Serp-1) treatment significantly reduced pristane induced lung hemorrhage (DAH) in C57Bl/6 mice, a mouse model for DAH. Gross pathology whole lung isolates at 14 days follow up. The figure shows lungs isolated from mice after euthanasia: treatment with saline daily X 14 days (top row), WT Serp-1 (second row) or PEG Serp-1 (Serp-1m5) (third row) given on the day of pristane injection and daily for 14 days after inducing DAH. Note that “modSerp-1^{m5}” and “Serp-1m5” may be used interchangeably. Treatment with WT Serp-1 or PEG-Sep-1 significantly reduced DAH. Treatment with PEG-Serp-1 starting 7 days after pristane (fourth row) or given for 7 days and then discontinued again (fifth row) markedly reduced lung hemorrhage at 14 days follow up. Dosage: 100 μg/kg (100 ng/gm body weight) WT or PEG-Serp-1 given by intraperitoneal (IP) injections.

FIG. 6C shows that modified Serp-1 proteins may reduce alveolar hemorrhage. For the data in this figure, lungs were stained with H&E. Red blood cells were stained bright pink, indicating alveolar hemorrhage. A DAH score was calculated based on the following scale: 0, no hemorrhage; 1, 0-25%; 2, 25-50%; 3, 50-75%; 4, 75-100%. The modified Serp-1 protein significantly reduced the DAH score.

FIG. 6D shows that modified Serp-1 proteins may reduce a macrophage response to hemosiderosis. For the data in this figure, lungs were stained with Prussian Blue, which stains iron breakdown (hemosiderin), especially in macrophages. Positive blue staining is indicative of an immune response to hemorrhage. Both the wild-type Serp-1 protein and the modified Serp-1 protein significantly reduced numbers of hemosiderin-laden macrophages.

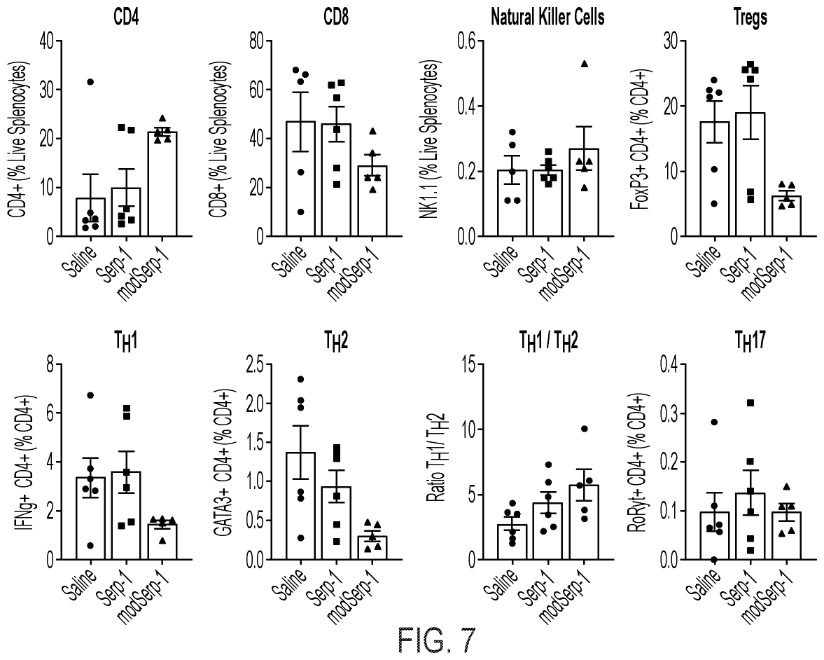

FIG. 7 further shows that modified Serp-1 proteins may reduce macrophage responses to hemosiderosis. Total splenocyte analysis by flow cytometry was undertaken. The data are indicative of administration of modified Serp-1 proteins promoting a CD4-biased splenocyte response, and that the modified Serp-1 proteins may more potently induce such a response than a wild-type Serp-1 protein.

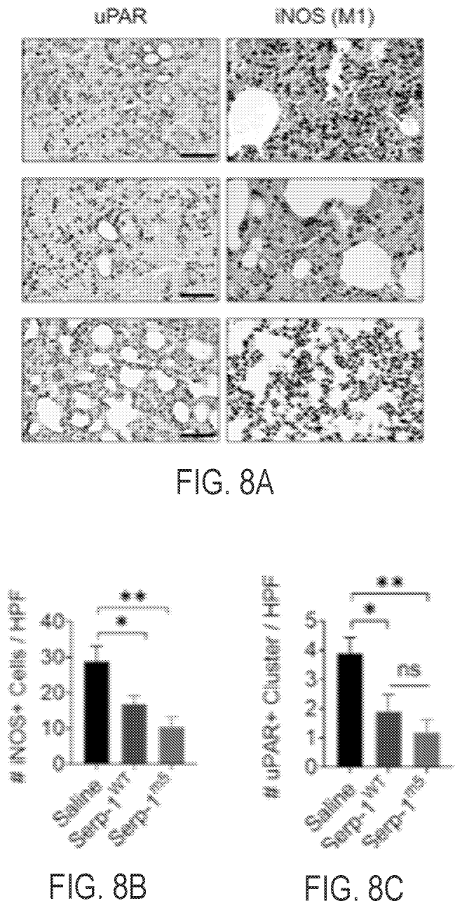

Modified Serp-1 proteins may improve alveolar tissue preservation. FIG. 8A shows that Serp-1m5 improved alveolar tissue preservation in immunohistochemistry (IHC) micrographs stained for uPAR, and M1 macrophage iNOS+staining. Bar graphs demonstrated that PEG Serp-1m5 and wild type Serp-1 (Serp-1WT) significantly reduced iNOS+M1 cell counts (FIG. 8B), and uPAR+stained clusters at 10 days follow up (FIG. 8C). *P<0.01; **P<0.001; dosage of 100 ng/gm body weight.

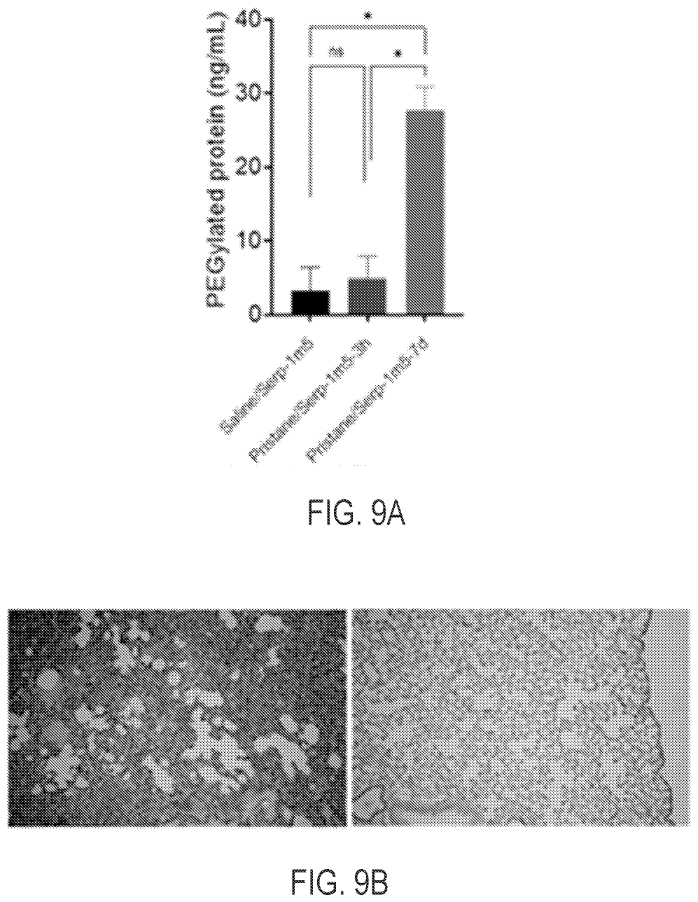

PEG-Serp-1 was detected only in hemorrhagic lungs of mice given pristane, demonstrating PEG-Serp-1 selective targeting of active proteases. Infused PEG-Serp-1 may bind to active proteases causing lung hemorrhage in the lungs of mice with pristane induced DAH. Both IHC and enzyme-linked immunosorbent assays (ELISAs) demonstrated increased PEG-Serp-1 in lungs of mice after PEG-Serp-1 (modSerp-1^{m5}) treatment. FIG. 9A includes graphical ELISA data showing detection of increased PEG-Serp-1 in pristane treated mice with lung hemorrhage, but not normal mice, without lung hemorrhage (DAH). FIG. 9B shows IHC data demonstrating increased PEG-Serp-1 staining in lungs from DAH mice (left panel), but not mice without pristane and DAH (right panel). 20× magnification was used.

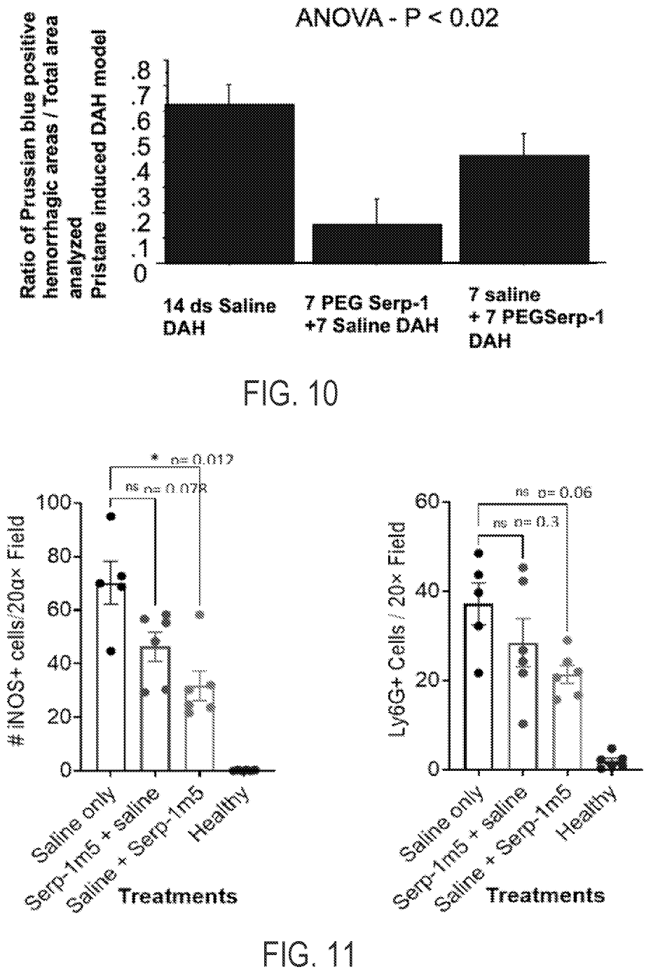

As seen in FIG. 10, Prussian blue staining for lung hemorrhage demonstrated significant decreases in percentage of hemorrhagic area—DAH score with 7 days PEG Serp-1 treatment P<0.0151 ANOVA (Fisher's PLSD P<0.0042 for 7 days PEG Serp-1 (modSerp-1^{m5}) followed by 7 days saline, P=0.1217 for 7 days delayed PEG Serp-1 treatment).

As seen with immunohistochemical staining, delayed PEG-Serp-1 (modSerp-1^{m5}) treatment reduced iNOS+M1 proinflammatory macrophage invasion significantly. A trend indicated a reduction in Ly6G+ cells. The left panel in FIG. 11 is a graph of IHC data demonstrating that with PEG Serp-1 treatment at 7 days after inducing DAH with pristane, again significantly reduced M1 macrophage invasion. The right panel in FIG. 11 includes Ly6G cell count data showing a non-significant trend toward reduced cell counts with early or later PEG Serp-1 treatments.

Overall, PEGylation of Serp-1 enhanced the in vivo bioactivity of Serp-1. These data indicate that the modified Serp-1 proteins may have biological activity in vivo. Likewise, other amounts or types of PEGylation (or other water soluble polymer) would be expected to confer similar enhanced effects on a Serp-1 protein. For example, a modified Serp-1 protein comprising a Serp-1 polypeptide conjugated to one or more therapeutic enhancing moieties comprising PEG molecules at various locations and having the following molecular weights are expected to also provide improved properties such as an in vivo biological activity of this Example: 5 kDa, 10 kDa, 15 kDa, 20 kDa, 25 kDa, 30 kDa, 35 kDa, or 40 kDa, or a range of molecular weights (e.g. 5-40 kDa, or a range that includes of any of the aforementioned weights).

### Example 4: Modified Serp-1 Bioactivity and Dose Range in a Preclinical DAH Model

PEGylated Serp-1 variants with the longest PK profile will be tested in the experimental pristane-induced DAH model. Briefly, the model will be induced by intraperitoneal injection of 500 μL pristane in female C57BL6/J mice per group at 6-8 weeks of age. Based on prior experiments and power analysis we will use 10 mice per group. Mice will be treated daily by intraperitoneal injections of saline alone or PEGylated Serp-1 variants at a dose of 100 ng/g bodyweight, or subcutaneous injections of dexamethasone at a dose of 2 μg/g as a comparison to the most common clinical treatment of corticosteroids. ModSerp-1^{m5} may be included as well. Treatment groups will be repeated in the absence of pristane induction. This initial dose is based on initial experiments as a dose wherein it is expected that at least bioactivity in modSerp-1^{m5} will be seen, and which will be considered the baseline in these experiments. After 14 days of treatment, mice will be euthanized, and tissues collected. Gross pathology will be used to score pulmonary hemorrhage and histopathology will be used to evaluate DAH score and hemosiderin-laden macrophage deposition as previously described. Blinded, board-certified pathologists will verify histopathologic scoring.

Variants which show equivalent or better efficacy, or other modulated properties, compared to modSerp-1^{m5} will be evaluated across the extended dose range of 300, 1000 and 3000 ng/g, providing a 5-point curve along with the prior 100 ng/g dose and saline control.

Mice will be observed daily and measurements of weight and clinical observation to evaluate activity and grooming capability will be performed as an incidental analysis of potential toxicity. Furthermore, histological assessment of the kidney, liver, heart, and spleen will be performed by H&E staining to determine potential effects on extrapulmonary tissues. This information will inform future toxicity testing.

### Example 5: PEG Serp-1 Testing in Pristane DAH Mouse Model

PEGylated Serp-1 or other modified forms of Serp-1 protein or peptide will be tested in the experimental pristane-induced DAH model for the evaluation of various modified forms of the protein or peptide. The model is induced by a single intraperitoneal injection of 500 μL pristane or saline (control) in male and female C57BL6/J mice per group at 8-12 weeks of age. Based on prior experiments and power analysis there are 20 mice per group as listed in Table 1. Mice are treated daily by intraperitoneal injections of therapeutic protein or peptide at a dose of 100 ng/g bodyweight immediately after pristane by intraperitoneal injection in 100 μL saline or with saline alone. For protein and peptide treatment, mice are IP injected with boluses daily until endpoint timing. Mice are euthanized at 5, 10 and 15 days post-DAH induction and tissue is collected in formalin for histology, frozen for western blot analysis, and serum collected for circulating cytokine analysis by ELISA. Survival is expected to be 70-80% at 15 days. Within the duration of the study, the mice are monitored and weighed daily. If any mice show signs of distress which include hunching, decreased mobility, vocalization, diarrhea, ulceration or dehiscence of the surgical incision, abdominal swelling or tenderness, decreased urine volume, infected wounds, limb ischemia, or weight loss of >1500 compared to pre-surgical weight, close monitoring and earlier sacrifice may be performed.

TABLE 1
Summary of the total number of mice required for Model

| Condition | Treatment | Dose | | Follow-up | | # mice* |
|---|---|---|---|---|---|---|
| 0.5 mL | Saline | 100 | μL | 5 | day | 20 |
| Pristane (i.p.) | | | | | | |
| | | | | 10 | day | 20 |
| | | | | 15 | day | 20 |
| | Serp-1 | 100 | ng/g | 5 | day | 20 |
| | protein | | | | | |
| | | | | 10 | day | 20 |
| | | | | 15 | day | 20 |
| | | | | 10 | day | 20 |
| | | | | 15 | day | 20 |
| 0.5 mL | Saline | 100 | μL | 5 | day | 20 |
| Saline (i.p.) | | | | | | |
| | | | | 10 | day | 20 |
| | | | | 15 | day | 20 |
| | Serp-1 | 100 | ng/g | 5 | day | 20 |
| | protein | | | | | |

| | | |
|---|---|---|
| 10 | day | 20 |
| 15 | day | 20 |

Total: 280
*As in some human cases, female mice exhibit worse symptoms after injection of pristane. We will perform experiments with both male and female mice and analyze the data both aggregated and sex-disaggregated. Ten mice of each sex are used for this experiments, but will initially only use 6 mice of each sex per prior power analysis for determining significance.

### Example 56: Biological Activity and Pharmacokinetics of Modified Serp-1 Proteins

A biological activity of modified Serp-1 proteins (including modSerp-1^{m5} and modSerp-1^{s10}) will be further assessed by determining the binding affinity between the modified Serp-1 proteins and uPA, and will be compared to a wild-type Serp-1 protein. Equilibrium dissociation constant (Kd) values will be determined using a binding assay.

Modified Serp-1 proteins will be purified by FPLC, characterized by both protein gel and specific modification sites will be identified by LC-MS/MS and CID-MS/MS.

Additional modified Serp-1 proteins will be prepared. Modified Serp-1 protein function will be assessed in vitro. This determination will be made in at least two ways. First, confirmation of serpin-enzyme complex formation will be performed by incubating the modified Serp-1 proteins in the presence of active recombinant uPA and evaluating the formation of the high molecular weight-shifted Serp-1:uPA complex. Second, the ability for the modified Serp-1 proteins to inhibit uPA activity will be measured in a quantitative kinetic assay. Briefly, uPA will be incubated in the presence of a 7-amino-4-trifluoromethylcoumarin (AFC)-conjugated fluorogenic substrate and the PEGylated Serp-1 variants, wildtype Serp-1 or without a serpin. Fluorescence generated by active uPA acting on the substrate and releasing free AFC will be measured in real-time on a fluorescence plate reader. Percent inhibition of uPA will be evaluated by a change in the kinetic growth curve of the fluorescent substrate.

Optimization of engineering and purification will also be performed. Specifically, the modification reaction will be optimized by testing a range of (i) temperature, (ii) time and (iii) molar ratio of Serp-1 and therapeutic enhancing moieties. Modified Serp-1 proteins will then be analyzed again by protein gel and mass spectrometry to confirm monomeric identity and modification site specificity. Each candidate variant in this sub-aim will be tested across 5 small-scale production batches. The optimal engineering and purification pipeline will be determined by which process produces the least variability in (i) yield, (ii) site-specificity of modification and (iii) percent monomeric identity across batches

Pharmacokinetic properties of modified Serp-1 proteins (including modSerp-1^{m5} and modSerp-1^{s10}) will be determined in mice, and compared to a wild-type Serp-1 protein. The in vivo half-life will be assessed. Overall characterization of PK properties of modified Serp-1 proteins will be performed. The circulating half-life and PK properties of the modified Serp-1 proteins will be evaluated in mice. Thirty female 6-8 week old C57BL6/J mice (N=3/timepoint) will be administered a single dose of a modified Serp-1 proteins or wildtype Serp-1 as an IV bolus injection at 100 ng/g. Blood samples be collected from three animals per timepoint at ten timepoints over 24 hours post-drug administration in EDTA collection tubes. Blood samples will be centrifuged to obtain plasma and stored at −80° C. until analysis by a ligand-binding assay. PK parameters will be estimated using Phoenix WinNonLin software (Cetera L. P., US) or GraphPad Prism. Parameters will be generated from mean group concentrations. The study will include 150 animals total (4 variants, 1 wildtype). PK parameters (half-life (t½), maximum concentration (Cmax), area under the plasma concentration-time curve (AUC), clearance (Cl)) will be used to further characterize the modified Serp-1 proteins and additional useful properties. PK study and analysis of collected plasma samples will be conducted by ligand-binding assay. PK parameters will be calculated.

In future studies, in vivo studies are conducted in mice, rats, dogs, cows, horses, chickens, cats, and any other animal species to characterize the pharmacokinetics (PK) of the modified Serp-1 proteins described herein after intravenous (IV) and/or subcutaneous (s.c.) dosing. In some embodiments, recombinant Serp-1 proteins comprising Serp-1 polypeptide mutant variants, modified Serp-1 proteins, PEGylated recombinant Serp-1, and/or Acylated recombinant Serp-1 proteins are observed to exhibit an effect on the in vivo half-life relative to wild-type Serp-1 proteins.

Formulation for some future in vivo studies: The Serp-1 protein test compound vehicle is PBS. Animal Dosing Design—In vivo PK, nonfasted animals Group 1: 3 animals per group+Control animals (for drug-free blood collection), n=2 rats. Plasma Sample Collection from rats (serial sampling): Blood aliquots (300 μL) are collected from jugular vein catheterized rats in tubes coated with lithium heparin, mixed gently, then kept on ice and centrifuged at 2,500×g for 15 minutes at 4° C., within 1 hour of collection. For control animals, blood is collected by cardiac puncture. The plasma is then harvested and kept frozen at −70° C. until further processing.

Quantitative Bioanalysis for Plasma in future studies: rat drug (modified Serp-1 protein) levels in plasma are measured by an ELISA. A plasma calibration curve is generated. Aliquots of drug-free plasma are spiked with the test compound at the specified concentration levels. The spiked plasma samples are processed together with the unknown plasma samples using the same procedure. The processed plasma samples are stored at −70° C. until the ELISA analysis, at which time the concentrations of the test compound in the unknown plasma samples are determined using the respective calibration curve. The reportable linear range of the assay is determined, along with the lower limit of quantitation.

Pharmacokinetics in future studies: plots of plasma concentration of modified and unmodified Serp-1 proteins versus time are constructed. Pharmacokinetic parameters of Serp-1 proteins after intravenous administration, including AUClast, AUCINF, T½, Cl, Vz, Vss, Tmax, and Cmax) are obtained from a non-compartmental analysis (NCA) of plasma data using WinNonlin. ELISAs are used to measure modified and unmodified Serp-1 protein concentration in serum.

Mice: in mice, the PK of the modified or unmodified Serp-1 proteins are determined following both i.v. (1 mg/kg) and s.c. (1 mg/kg) administration. Three mice are bled at each time point and serum samples are analyzed by an ELISA or by a Serp-1 activity assay.

Rats: Sprague-Dawley rats are also dosed with modified or unmodified Serp-1 proteins (i.v., 1 mg/kg; s.c., 1 mg/kg) and PK profiles are determined. Three rats are bled at each time point and serum samples are analyzed by an ELISA or by a Serp-1 activity assay.

Beagle dogs: in beagles, the PKs of the modified or unmodified Serp-1 proteins are determined following both i.v. (1 mg/kg) and s.c. (1 mg/kg) administration. Two dogs are bled at each time point after i.v. dosing and one dog per dose group is bled after s.c. dosing.

Other corresponding species: in relevant animal species (e.g. cow, pig, horse, sheep, dog, cat, chicken) the PKs of modified or unmodified Serp-1 proteins are determined following both i.v. (1 mg/kg) and s.c. (0.2 mg/kg) administration. Two or more animals are bled at each time point and serum samples are analyzed by an ELISA or by a Serp-1 activity assay.

### Example 7: PK Analysis of PEG Serp-1 (pK/pD)

The PEGylated Serp-1 half-life is tested in the experimental mouse model. As detailed in Table 2, C57Bl/6 mice are treated with 100 μL of WT Serp-1 (100 microgram/kg; 0.1 mg/kg) or 100 microL of PEG Serp-1 at one of two doses via tail vein injection (100 microgram/kg, 0.1 mg/kg; or 500 μg/kg, 0.5 mg/kg) at time 0. pK is measured for each individual dose as recommended by our consultant on pK/pD analyses. Cardiac puncture is performed to withdraw 300 μL of blood per mouse at time points of 3, 10, 30, 60 min, 2, 4, 6, 8, 12, 24 hours post-dose. Blood is drawn up into heparinized syringes for plasma. A cohort or 10 mice is used as untreated controls. Mice are euthanized at the time of cardiac puncture. No other drugs are given. No other procedures are performed. For all follow up times, mice have cardiac puncture immediately after CO2 euthanasia. The half life is measured in the original GMP Serp-1 product used for clinical trials, indicates a circulating half life of 20 minutes thus an early time point at 3 minutes is suggested by our consultant. Further study details are listed in Table 3.

One hundred additional C57BL/6 mice are ordered from JAXLabs; 3-6 mice per drug and dose injection of Serp-1 or PEG Serp-1 and per follow up times. Power calculation and prior research demonstrates that three mice per time of follow up and treatment is acceptable to perform the pK analysis for PEGSerp-1 based upon the prior pK study performed with the WTSerp-1. The number of mice is based upon standard pK studies as performed originally with our original pK studies performed with WT Serp-1 prior to FDA approval and used for preclinical safety and clinical trials for the clinical Phase 1 and Phase 2 a Trials. We use the minimal number of mice (3 per dose and treatment and time to follow up) for this pK study for PEGSerp-1, but we have provided for an additional 100 mice if larger numbers are necessary for detection of circulating PEGSerp-1.

TABLE 2
Half-life of PEG Serp-1 study dosage

| | | Route of | Dose | | No. Mice |
|---|---|---|---|---|---|
| Group | Test Article | Administration | mL | mg/kg | (Male) |
| 1 | Serp-1 [PEG] | IV | 0.1 mL | 0.1 | 30^{a} + 3^{b} |
| 2 | Serp-1 [PEG] | IV | 0.1 mL | 0.5 | 30^{a} + 3^{b} |
| 3 | WT Serp-1 | IV | 0.1 mL | 0.5 | 30^{a} + 3^{b} |
| | VT-111 | | | | |
| | [clinical lot] | | | | |
| 4 | PBS control | IV | 0.1 mL | 0 | 9 |

^{a}1/timepoint
^{b}untreated (blank) control −10controls

TABLE 3
PEG Serp-1 PK study protocol
Animal Use

| | |
|---|---|
| Animal Species/Strain | Mice, C57B1/6 |
| Animal Weight Range (g) | 20-30 |
| Fasting Regimen | None |
| Pre-dose Observations | Body weight (report) |

Test Article Information and PK Sample Collection

| | |
|---|---|
| Test Article ID: | Serp-1 [PEG] |
| | VT-111 [clinical lot] |
| Vehicle/Formulation: Sterile PBS | |
| Total number of animals: | 100 (1/timepoint/Serp-1 |
| | injection and dose) |
| Total number of samples: | 100 |
| PK sample collection times | 3, 10, 30, 60 |
| | min, 2, 4, 6, 8, 12, 24 |
| | hours post-dose |
| Target blood sample volume (ml) | No less than 0.3 ml |
| | via cardiac puncture |
| Anticoagulant | K2EDTA or heparin |
| Sample storage | −80° C. |

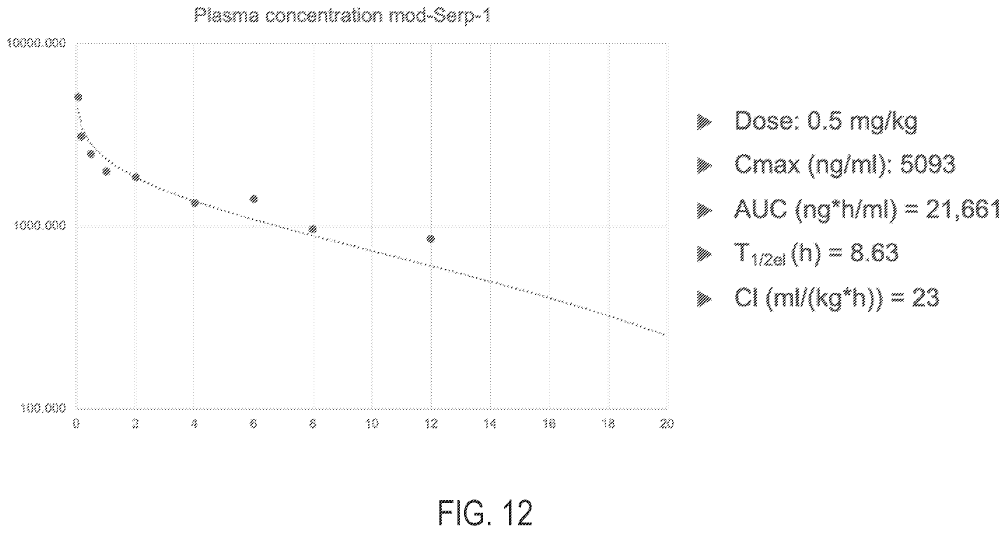

As shown in FIG. 12, a circulating half-life in an initial analysis appeared improved with a PEGylated Serp-1 (modSerp-1^{m}) compared to unmodified Serp-1. A longer circulating blood half-life was seen in mice, with an increase from 20-30 minutes for wild type (WT) Serp-1 to a half-life for PEGylated Serp-1 up to about 8-9 hours. Thus a modified Serp-1 (e.g. with a therapeutic enhancing moiety) may have an increase in half-life compared to unmodified Serp-1 by about 16-fold to 27-fold. The pK analysis of the modified Serp-1 in FIG. 12 shows an increased circulating PEGylated Serp-1 half life in C57Bl/6 mice, where t1/2 was calculated as 8.63 hours

What is claimed is:

1. A modified Serp-1 protein comprising at least one therapeutic enhancing moiety, wherein the modified Serp-1 protein is biologically active.

2. The modified Serp-1 protein of claim 1, comprising a polypeptide comprising a sequence having at least 80% sequence identity to SEQ ID NO: 1, or a fragment thereof.

3. The modified Serp-1 protein of claim 2, wherein the polypeptide is encoded by a nucleic acid.

4. The modified Serp-1 protein of claim 3, wherein the therapeutic enhancing moiety is encoded by the nucleic acid.

5. The modified Serp-1 protein of claim 2, wherein the polypeptide comprises one, two, three, four or more amino acid substitutions, insertions, or deletions, wherein the substitutions are with natural or non-naturally encoded amino acids.

6. The modified Serp-1 protein of claim 1, wherein the therapeutic enhancing moiety comprises a pharmacokinetic enhancing moiety, a stability enhancing moiety, a thermal stability enhancing moiety, or an activity enhancing moiety.

7. The modified Serp-1 protein of claim 1, wherein the modified Serp-1 protein has enhanced therapeutic effects, enhanced pharmacokinetics, enhanced stability, enhanced thermal stability, or enhanced activity, compared to an unmodified or wild-type Serp-1 protein.

8. The modified Serp-1 protein of claim 1, wherein the therapeutic enhancing moiety comprises a hydrophilic molecule, a PEGylation, an acyl group, a lipid, an alkyl group, a carbohydrate, a polypeptide, a polynucleotide, a polysaccharide, an antibody or antibody fragment, a sialic acid, a prodrug, a serum albumin, an XTEN molecule, an Fc molecule, adnectin, fibronectin, a biologically active molecule, or a water soluble polymer, or a combination thereof.

9. The modified Serp-1 protein of claim 1 wherein the therapeutic enhancing moiety is a water soluble polymer comprising polyethylene glycol, polyethylene glycol propionaldehyde, mono C1-C10 alkoxy or an aryloxy derivative thereof, polyethylene glycol, polyvinyl pyrrolidone polyvinyl alcohol, a polyamino acid, divinylether maleic anhydride, N-(2-Hydroxypropyl)-methacrylamide, dextran, a dextran derivative, dextran sulfate, polypropylene glycol, polypropylene oxide copolymer, polyoxyethylated polyol, heparin, a heparin fragment, a polysaccharide, an oligosaccharide, a glycan, cellulose, a cellulose derivative, methylcellulose, carboxymethyl cellulose, starch, a starch derivative, a polypeptide, polyalkylene glycol or a derivative thereof, a copolymer of polyalkylene glycol or a derivative thereof, a polyvinyl ethyl ether, or alpha-beta-poly[(2-hydroxyethyl)-DL-aspartamide, or a combination thereof.

10. The modified Serp-1 protein of claim 1, wherein the therapeutic enhancing moiety comprises or consists of a water soluble polymer.

11. The modified Serp-1 protein of any one of claims 1-10, wherein the therapeutic enhancing moiety comprises polyethylene glycol (PEG).

12. The modified Serp-1 protein of claim 11, wherein the PEG is branched.

13. The modified Serp-1 protein of claim 11, wherein the PEG is unbranched.

14. The modified Serp-1 protein of claim 1, wherein the therapeutic enhancing moiety comprises at least one acyl group, or at least one alkyl group.

15. The modified Serp-1 protein of claim 1, wherein the therapeutic enhancing moiety has a molecular weight of about 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, 150 Da, 100 Da, 75 Da, or 57 Da, or a range of molecular weights defined by any two of the aforementioned molecular weights.

16. The modified Serp-1 protein of claim 1, wherein the at least one therapeutic enhancing moiety has a molecular weight of about 5 kDa.

17. The modified Serp-1 protein of claim 1, wherein the at least one therapeutic enhancing moiety has a molecular weight of about 10 kDa.

18. The modified Serp-1 protein of claim 1, wherein the therapeutic enhancing moiety is conjugated to a naturally occurring or non-naturally occurring amino acid of the polypeptide.

19. The modified Serp-1 protein of claim 1, wherein the therapeutic enhancing moiety is linked to a lysine of the polypeptide.

20. The modified Serp-1 protein of claim 1, wherein the therapeutic enhancing moiety is linked to a cysteine of the polypeptide.

21. The modified Serp-1 protein of claim 1, wherein the therapeutic enhancing moiety is chemically conjugated to a site at or near an N-terminus or C-terminus of the polypeptide.

22. The modified Serp-1 protein of claim 1, wherein the therapeutic enhancing moiety is linked to an end of the polypeptide.

23. The modified Serp-1 protein of claim 1, wherein the therapeutic enhancing moiety is linked to an amino terminus of the polypeptide.

24. The modified Serp-1 protein of claim 1, wherein the therapeutic enhancing moiety is linked to a carboxyl terminus of the polypeptide.

25. The modified Serp-1 protein of claim 1, wherein the therapeutic enhancing moiety is randomly conjugated to the polypeptide.

26. The modified Serp-1 protein of claim 1, wherein the therapeutic enhancing moiety is connected to the polypeptide through a linker.

27. The modified Serp-1 protein of claim 1, wherein the therapeutic enhancing moiety comprises at least one additional Serp-1 protein or modified Serp-1 protein.

28. The modified Serp-1 protein of claim 1, wherein the therapeutic enhancing moiety is linked to multiple Serp-1 proteins.

29. The modified Serp-1 protein of claim 2, wherein the therapeutic enhancing moiety is covalently connected to the polypeptide.

30. The modified Serp-1 protein of claim 1, wherein the Serp-1 protein is cross-linked with multiple Serp-1 proteins.

31. The modified Serp-1 protein of claim 1, wherein the at least one therapeutic enhancing moiety comprises 1, 2, 3, 4, 5, 6, 7, 8, 9, 10, 11, 12, 13, 14, 15, 16, 17, 18, 19, 20, 21, 22, 23, 24, 25, or more therapeutic enhancing moieties, or a range of therapeutic enhancing moieties defined by any two of the aforementioned integers.

32. The modified Serp-1 protein of claim 1, wherein the polypeptide is produced by a cell.

33. The modified Serp-1 protein of claim 32, wherein the polypeptide is secreted from the cell.

34. The modified Serp-1 protein of claim 32, wherein the cell is a prokaryotic cell.

35. The modified Serp-1 protein of claim 32, wherein the cell is a eukaryotic cell.

36. The modified Serp-1 protein of claim 35, wherein the eukaryotic cell is a mammalian cell.

37. The modified Serp-1 protein of claim 32, wherein the cell comprises a cell line.

38. The modified Serp-1 protein of claim 37, wherein the cell line comprises a CHO cell.

39. The modified Serp-1 protein of claim 32, wherein the cell comprises a human cell.

40. The modified Serp-1 protein of claim 1, wherein the modified Serp-1 protein is purified or is substantially pure.

41. The modified Serp-1 protein of claim 1, wherein the modified Serp-1 protein is purified from the cell or from cell media.

42. The modified Serp-1 protein of claim 1, wherein the modified Serp-1 protein exhibits an in vivo half-life that is greater than an unmodified Serp-1 protein.

43. The modified Serp-1 protein of claim 42, wherein the unmodified Serp-1 protein comprises the polypeptide.

44. The modified Serp-1 protein of claim 42, wherein the unmodified Serp-1 protein exhibits an in vivo half-life of at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 1 week, at least 2 weeks, at least 3 weeks, at least 4 weeks, or longer.

45. The modified Serp-1 protein of claim 42, wherein the in vivo half-life is determined in a subject comprising an animal, a vertebrate, a mammal, a rodent, a dog, a rabbit, a horse, cattle, a cat, a sheep, a chicken, a pig, a primate, a non-human primate, or a human.

46. The modified Serp-1 protein of claim 42, wherein the half-life is measured in a mammal.

47. The modified Serp-1 protein of claim 42, wherein the half-life is measured in a human.

48. The modified Serp-1 protein of claim 1, wherein the modified Serp-1 protein is stable at a temperature of 25° C., 30° C., 35° C., 40° C., 45° C., 50° C., 55° C., 60° C., 65° C., 70° C., 750 C, 80° C., 85° C., 90° C., 95° C., 100° C., or more, or a range of temperatures defined by any two of the aforementioned temperatures.

49. The modified Serp-1 protein of claim 48, wherein the stability lasts at least 1 minute, at least 2 minutes, at least 3 minutes, at least 4 minutes, at least 5 minutes, at least 10 minutes, at least 15 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 1 week, at least 2 weeks, at least 3 weeks, at least 4 weeks, or longer.

50. The modified Serp-1 protein of claim 1, wherein the modified Serp-1 protein exhibits an in vitro thermal stability that is greater than an unmodified Serp-1 protein.

51. The modified Serp-1 protein of claim 50, wherein the unmodified Serp-1 protein comprises the polypeptide.

52. The modified Serp-1 protein of claim 1, wherein the modified Serp-1 protein is attached to another biologically active moiety.

53. The modified Serp-1 protein of claim 1, wherein the modified Serp-1 protein includes at least one, at least two, or three additions, deletions, or substitutions of amino acids of a mature wild-type Serp-1 protein.

54. The modified Serp-1 protein of claim 2, wherein the polypeptide comprises a mature wild-type Serp-1 protein.

55. The modified Serp-1 protein of claim 1, wherein the biological activity of the modified Serp-1 protein comprises binding to u-plasminogen activator (uPA).

56. The modified Serp-1 protein of claim 55, wherein the binding between the modified Serp-1 protein and uPA comprises a binding affinity with an equilibrium dissociation constant (Kd) below 1 mM, below 750 μM, below 500 μM, below 250 μM, below 200 μM, below 150 μM, below 100 μM, below 75 μM, below 50 μM, a Kd below 45 μM, a Kd below 40 μM, a Kd below 35 μM, a Kd below 30 μM, a Kd below 25 μM, a Kd below 20 μM, a Kd below 15 μM, a Kd below 14 μM, a Kd below 13 μM, a Kd below 12 μM, a Kd below 11 μM, a Kd below 10 μM, a Kd below 9 μM, a Kd below 8 μM, a Kd below 7 μM, a Kd below 6 μM, a Kd below 5 μM, a Kd below 4 μM, a Kd below 3 μM, a Kd below 2 μM, or a Kd below 1 μM.

57. The modified Serp-1 protein of claim 1, wherein the modified Serp-1 protein is conjugated to at least one of a label, a dye, a polymer, a water-soluble polymer, a photocrosslinker, a radionuclide, a cytotoxic compound, a drug, an affinity label, a photoaffinity label, a reactive compound, a resin, another polypeptide or protein, a polypeptide analog, an antibody, an antibody fragment, a metal chelator, a cofactor, a fatty acid, a carbohydrate, a polynucleotide, a DNA, an RNA, an antisense polynucleotide, a saccharide, a water-soluble dendrimer, a cyclodextrin, an inhibitory ribonucleic acid, a biomaterial, a nanoparticle, a spin label, a fluorophore, a metal-containing moiety, a radioactive moiety, a functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, a quantum dot, a nanotransmitter, a radionucleotide, a radiotransmitter, or a neutron-capture agent, or a combination thereof.

58. A culture medium, or an isolated cell, vector, plasmid, prokaryotic cell, eukaryotic cell, virus, AAV, mammalian cell, yeast, bacterium, or cell-free translation system comprising the modified Serp-1 protein of any one of claims 1-57.

59. A composition comprising the culture medium, or isolated cell, vector, plasmid, prokaryotic cell, eukaryotic cell, virus, AAV, mammalian cell, yeast, bacterium, or cell-free translation system of claim 58, and a pharmaceutically acceptable carrier.

60. A composition comprising the modified Serp-1 protein of any of claims 1-57, and a pharmaceutically acceptable carrier.

61. The composition of claim 59, wherein the pharmaceutically acceptable carrier comprises a buffer.

62. The composition of claim 59, further comprising one or more other active compounds comprising a drug, a vaccine, an antibiotic, an antiviral compound, or an anti-parasitic compound.

63. A method, comprising administering the composition of claim 60 to a subject.

64. The method of claim 63, wherein the subject an animal, a vertebrate, a mammal, a rodent, a dog, a rabbit, a horse, cattle, a cat, a sheep, a chicken, a pig, a primate, or a non-human primate.

65. The method of claim 63, wherein the subject is a mammal.

66. The method of claim 63, wherein the subject is a human.

67. A modified Serp-1 protein or composition containing the modified Serp-1 protein of claim 1, for use as a medicament.

68. Use of a modified Serp-1 protein or composition containing the modified Serp-1 protein of claim 1 for the manufacture of a medicament for treating or preventing a disease or disorder.

69. An expression cassette comprising a nucleic acid encoding the modified Serp-1 protein of claim 1.

70. The expression cassette of claim 69, wherein the nucleic acid comprises DNA.

71. The expression cassette of claim 69, configured for expression in a cell.

72. The expression cassette of claim 71, wherein the cell comprises a mammalian cell.

73. The expression cassette of claim 71, wherein the cell is a CHO cell.

74. The expression cassette of claim 71, wherein the cell is a human cell.
